(19) United States
(12) Patent Application Publication
(10) Pub. No.: US 2010/0112563 A1
(43) Pub. Date: May 6, 2010

(54) MULTIPLEX ANALYSIS OF NUCLEIC ACIDS

(75) Inventors: Wen-Tso Liu, Singapore (SG); Jer-Horng Wu, Taiwan (CN)

Correspondence Address: OSHA LIANG L.L.P., TWO HOUSTON CENTER, 909 FANNIN, SUITE 3500, HOUSTON, TX, 77010, US

(73) Assignee: NATIONAL UNIVERSITY OF SINGAPORE, Singapore (SG)

(21) Appl. No.: 12/376,185

(22) PCT Filed: Aug. 3, 2007
(86) PCT No.: PCT/SG07/00231

§ 371 Date: Jun. 8, 2009

Related U.S. Application Data
(60) Provisional application No. 60/835,649, filed on Aug. 3, 2006.

(51) Int. Cl. C12Q 1/68

(52) U.S. Cl. 435/6

(57) ABSTRACT

A method for identifying target nucleic acids includes the steps of contacting a sample containing a plurality of target nucleic acids with at least one series of nucleotide primers under conditions that allow binding of said primers to at least one of said target nucleic acids and labeling of said bound primers with a detectable signal, wherein one member within each series has a lower level of specificity than other members of the series; and measuring said detectable signal of each labeled primer to determine the identity of said target nucleic acids.

### TECHNICAL FIELD

The present invention generally relates to methods for the detection, identification and quantification of target nucleic acids in a sample. In particular, the present invention relates to multiplex methods using a plurality of hierarchical oligonucleotide primers to detect, identify and quantify target nucleic acids in a sample, such as an environmental or a biological sample.

### BACKGROUND

Numerous molecular methods have been developed for the analysis of a sample containing multiple targets. Such methods are used in applications ranging from microbial community analysis, microbial monitoring, and microbial identification to identification of diseases in patients based on disease-related biomarkers. However, such methods vary in detection sensitivity and threshold, quantification capability, ease of use, and cost. For example, whilst the 16S rRNA gene based clone, library approach is able to identify numerically dominant microbial populations down to species or strain level, in a given ecosystem, it is a time-consuming and costly method. Similarly, whilst community fingerprinting techniques like denaturing gradient gel electrophoresis (DGGE) and terminal restriction fragment length polymorphism (T-RFLP) is able to rapidly profile dominant microbial populations down to approximately 0.1% of the total PCR-amplified products in a microbial community structure and allow comparison of different microbial community structures obtained from different environments or at different times and locations in a same environment, such fingerprinting methods are unable to identify individual species in a given microbial community without prior knowledge of the species that are present and the results are often influenced by the inconsistency associated with DNA extraction and PCR amplification.

Other methods have also been developed to identify specific types of microbial populations in a microbial community, for example whole-cell fluorescence in situ hybridization (FISH), membrane hybridization and real-time quantitative PCR (qPCR), and sequence-specific enzymatic cleavage methods. However, FISH requires lengthy fixation and hybridization procedures, and can be influenced by the poor penetration of probes through cell membranes, the low copies of rRNA inside inactive cells, and heterogeneous abiotic materials present in an ecosystem. With membrane hybridization and qPCR, standard curves have first to be established and optimized using reference strains, and hence, are not only labor-intensive and time-consuming, but are also incapable of high throughput applications.

For high throughput applications, for example in the detection of microorganisms and monitoring of microbial community structures, microarray technology currently provides the most accurate measurements. However, it is currently difficult to conduct multiplex analysis on microarrays in large-scale analysis of microbial abundance.

Another method recently developed utilizes sequence-specific enzymatic cleavages to quantify 16S rRNA targets of interest. The abundance of 16S rRNA targets is evaluated by comparing the peak areas (intensity) of cleaved 16S rRNA to that of total 16S rRNA in the electrophoregram. Whilst this technique does not require the prior preparation of an external standard curve or the use of an internal standard, it can only analyze one 16S rRNA target in each experiment.

There is a need to provide a rapid, high throughput, sensitive and accurate method with high reproducibility for determining the presence, identity and/or quantity of target nucleic acids in a sample that overcomes, or at least ameliorates, one or more of the disadvantages described above.

### SUMMARY

According to a first aspect, there is provided a method for identifying target nucleic acids comprising the steps of
(a) contacting a sample containing a plurality of target nucleic acids with at least one series of nucleotide primers under conditions that allow binding of said primers to at least one of said target nucleic acids and labeling of said bound primers with a detectable signal, wherein one member within each series has a lower level of specificity than other members of the series; and
(b) measuring said detectable signal of each labeled primer to determine the identity of said target nucleic acids.

In one embodiment, the sample comprises amplified target nucleic acids.

In one embodiment, the target nucleic acids comprise 16S ribosomal RNA.

In one embodiment, the method of the first aspect further comprises the step of purifying the labeled primers prior to step (b). The purification step may comprise chromatographic separation, enzyme-based digestion, spin column purification, chemical precipitation or electrophoresis separation.

In one embodiment, each series comprises between 3 and 20 nucleotide primers. At least one of the nucleotide primers within a series may have a different length to other members of the series. Alternatively, each nucleotide primer within a series may have a different length to other primers of the series.

The different lengths may comprise differences of from about 2 to about 70 nucleotides and may be obtained using nucleic acid tails of different lengths. Exemplary nucleic acid tails include poly dA tails and poly dT tails. The nucleic acid tails for individual members of a series may range from about 2 to about 50 nucleotides.

In one embodiment, after a nucleotide primer is bound to a target nucleic acid, the method comprises the step of extending the primer with a single nucleotide labeled with a detectable signal. Exemplary single nucleotides include the dideoxynucleoside triphosphates ddATP, ddTTP, ddCTP and ddGTP. The detectable signal may be a fluorescent signal, a chemiluminescent signal, a luminescent signal, a radioactive signal or a biotinylated signal. Exemplary fluorescent signals include fluorescein, rhodamine, coumarin, cyanine, fluorescent nanoparticles and fluorescent quantum dyes.

In one embodiment, the detectable signal of a labeled primer is quantified by application of a calibration factor of one or more target nucleic acids. The calibration factor may be obtained by determining the ratio of the intensity of a detectable signal of a bound primer having a higher level of specificity relative to the intensity of a detectable signal of a bound primer having a lower level of specificity.

In one embodiment, the method of the first aspect comprises, prior to step (b), the step of separating labeled primers to enable determination of the identity of target nucleic acids. Separation may be conducted using electrophoresis, chromatography or mass spectrometry.

In one embodiment, the conditions that allow binding of primers to at least one target nucleic acid and labeling of the bound primers with a detectable signal comprise conditions for annealing of the primers to the target nucleic acids and extension of the primers with at least one single nucleotide labeled with a detectable signal, and multiple cycles thereof.

In one embodiment, step (a) of the method of the first aspect comprises contacting a first aliquot of a sample with a first series of nucleotide primers and separately contacting a second aliquot of the sample with a second series of nucleotide primers, wherein the first and second series include at least one nucleotide primer in common.

In one embodiment, the method is a method of determining constituent organisms in a heterogenous sample. The constituent organisms may be prokaryotes such as bacteria and archae or eukaryotes such as fungi and yeast.

In one embodiment, the series of nucleotide primers comprises a plurality of nucleotide primers having a hierarchical level of specificity. The series may comprise a primer at a higher hierarchical level and a plurality of primers at a lower hierarchical level.

In one embodiment, the series of nucleotide primers comprises a plurality of species-specific primers and a non-species-specific primer. The series may comprise a domain-specific primer and a plurality of primers selected from the group consisting of phylum-specific primers, class-specific primers, order-specific primers, family-specific primers, genus-specific primers and species-specific primers, or may comprise a phylum-specific primer and a plurality of primers selected from the group consisting of class-specific primers, order-specific primers, family-specific primers, genus-specific primers and species-specific primers, or may comprise a class-specific primer and a plurality of primers selected from the group consisting of order-specific primers, family-specific primers, genus-specific primers and species-specific primers, or may comprise an order-specific primer and a plurality of primers selected from the group consisting of family-specific primers, genus-specific primers and species-specific primers, or may comprise a family-specific primer and a plurality of primers selected from the group consisting of genus-specific primers and species-specific primers, or may comprise a genus-specific primer and a plurality of species-specific primers.

In one embodiment, each member of a series, other than the one member with the lower level of specificity, is specific for one target nucleic acid.

In one embodiment, at least one nucleotide primer within a series is capable of binding to multiple target nucleic acids.

According to a second aspect, there is provided a kit for use in a method for identifying target nucleic acids, the kit comprising:
(i) at least one series of nucleotide primers capable of binding to a plurality of target nucleic acids, wherein one member within each series has a lower level of specificity than other members of the series; and
(ii) instructions for contacting a sample containing a plurality of nucleic acids with the nucleotide primers under conditions that allow binding of the primers to at least one of the target nucleic acids and labeling of the bound primers with a detectable signal to allow measurement of the detectable signal of each labeled primer to determine the identity of the target nucleic acids.

In one embodiment, the kit further comprises a set of labels having a detectable signal capable of binding to the nucleotide primers.

In one embodiment, at least one of the nucleotide primers within a series has a different length to other members of the series.

In one embodiment, each nucleotide primer within a series has a different length to other members of the series.

According to a third aspect, there is provided a method for diagnosis of a disorder of the gut in a subject, wherein said disorder is associated with an abnormal distribution of gut flora, said method comprising identifying target nucleic acids in a sample from the subject, the identifying comprising the steps of:
(a) contacting the sample containing target nucleic acids with at least one series of nucleotide primers under conditions that allow binding of the primers to at least one of the target nucleic acids and labeling of the bound primers with a detectable signal, wherein one member within each series has a lower level of specificity than other members of the series; and
(b) measuring the detectable signal of each labeled primer to determine the identity of the target nucleic acids,
   wherein the identification provides a determination of gut flora distribution.

### DEFINITIONS

The following words and terms used herein shall have the meaning indicated:

The term “multiplex” refers to multiple reactions being undertaken in a single reaction vessel.

The term “plurality” as used herein means at least two.

The term “nucleic acid”, and equivalent terms such as “polynucleotide”, refers to a polymeric form of nucleotides of any length, such as ribonucleotides (RNA), deoxyribonucleotides (DNA) or peptide nucleic acids (PNAs), that comprise purine and pyrimidine bases, or other natural, chemically or biochemically modified, non-natural, or derivatized nucleotide bases. The nucleic acid may be double stranded or single stranded. The backbone of the polynucleotide may comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The terms nucleoside, nucleotide, deoxynucleoside and deoxynucleotide generally include complements, fragments and variants of the nucleoside, nucleotide, deoxynucleoside and deoxynucleotide, or analogs thereof. Polynucleotides include “oligonucleotides” which typically comprise 2 to about 500 nucleotides.

The term “primer” as used herein means a single-stranded oligonucleotide capable of binding to a target nucleic acid. Typically, the binding is selective binding. The precise length of a primer will vary according to the particular application, but typically ranges from about 15 to about 120 nucleotides. A primer need not reflect the exact sequence of the target nucleic acid template but must be sufficiently complementary to bind to the template. The term “nucleotide primers” includes “oligonucleotide primers”.

The term “series” refers to a set of nucleotide primers that is used in a single reaction vessel of a multiplex reaction. Within each series, the nucleotide primers may be of the same or different lengths, and may be coupled to labels emitting the same or different detectable signals, and may have different levels of specificities, with some primers being able to bind to multiple target nucleic acids, and some being able to bind only one specific target nucleic acid.

The term “species-specific nucleic acid” refers to nucleic acid derived from a specific species of organism. In some embodiments, species-specific nucleic acids derived from a first organism may be present in a mixed sample that comprises species-specific nucleic acids derived from a second organism. The terms “domain-specific nucleic acid”, “phylum-specific nucleic acid”, “class-specific nucleic acid”, “order-specific nucleic acid”, “family-specific nucleic acid” and “genus-specific nucleic acid” shall be construed accordingly.

The term “selectively binds” refers to the ability of a nucleotide primer to preferentially bind to a target nucleic acid. In indicating that a nucleotide primer “selectively binds”, the term includes reference to binding, under stringent binding conditions, of the nucleotide primer to a target nucleic acid to a detectably greater degree than to a non-target nucleic acid.

The term “detectable label” refers to a reporter molecule or enzyme that is capable of generating a detectable and measurable signal, and may be covalently or non-covalently joined to a nucleotide primer.

The term “labeled”, when used in connection with a nucleotide primer, is intended to encompass direct labeling of the primer by covalently or non-covalently coupling (for example, physically linking) a detectable substance (for example, a fluorescent label) to the primer, as well as indirect labeling of the primer by reactivity with another reagent that is directly labeled. Direct labeling may be achieved with single nucleotide chain extension, which comprises the extension of a nucleotide primer with a single nucleotide that is labeled with a detectable signal such as a fluorescent label. Examples of indirect labeling include detection of a primary antibody using a fluorescently labeled secondary antibody and end-labeling of a DNA primer with biotin such that it can be detected with fluorescently labeled streptavidin.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DISCLOSURE OF OPTIONAL EMBODIMENTS

Exemplary, non-limiting embodiments of a novel method for detecting, identifying and/or quantifying a plurality of target nucleic acids will now be disclosed.

There is provided a multiplexing method, hierarchical oligonucleotide primer extension (HOPE), for rapid detection, identification and/or quantification of a plurality of different target nucleic acids in a sample containing a mixture of different nucleic acids. Plurality in this context means at least two.

The sample may be any sample, at least some constituents of which are to be determined. For example, the sample may be an environmental sample, a biological sample, or a food sample.

An environmental sample includes, but is not limited to, a sample obtained or derived from soil, water, wastewater, mud, vegetal extract, wood, biological material, marine or estuarine sediment, industrial effluents, gas, mineral extracts, sand, natural excrements, meteorites etc. The sample may be collected from various regions or conditions, such as tropical regions, deserts, volcanic regions, forests, farms, industrial areas, household, etc.

A biological sample refers to a sample obtained from an organism or from components (such as cells) of an organism. The sample may be of any biological tissue or fluid. Typically, the biological sample is a “clinical sample” derived from a patient. Such samples include, but are not limited to, feces, fecal matter, stools, sputum, cerebrospinal fluid, blood, blood fractions (e.g. serum, plasma), blood cells (e.g. white cells), tissue or fine needle biopsy samples, urine, peritoneal fluid, and pleural fluid, or cells therefrom as well as sections of tissues such as frozen sections taken for histological purposes.

A food sample refers to a substance that may be ingested by an animal, for example a human, and includes packaged food products, dairy products, animal products, vegetable products, raw food material, un-pasteurized or pasteurized food material, and water.

The sample may be untreated, treated, diluted or concentrated. The sample may be analyzed directly or may be subject to some preparation prior to use in the disclosed methods. Such preparation includes, but is not limited to, suspension/dilution of the sample in water or an appropriate buffer or removal of cellular debris or other undesired components, for example by centrifugation, or selection of particular fractions of the sample before analysis.

The target nucleic acids may be any nucleic acids that are to be sorted out from other nucleic acids in a sample containing a mixture of nucleic acids. The target nucleic acids may be RNA, DNA, cDNA or PNA. The target nucleic acids may be different regions of the same nucleic acid molecule. For example, the target nucleic acids may comprise a specific gene cluster such as 16S rRNA, or other functional genes.

Where desired, the target nucleic acids may be extracted from the sample using techniques known in the art, for example, by sonication, treatment with detergent, enzymatic digestion, salt precipitation, alcohol precipitation, and phenol-chloroform extraction.

The target nucleic acids, for example, genomic DNA and native RNA, may be used directly in the disclosed methods. In some embodiments, the target nucleic acids may be amplified to produce additional copies of some or all of the target nucleic acids prior to use in the disclosed methods. For example, target nucleic acids may be amplified using PCR technologies (see Sambrook et al., Molecular Cloning: A Laboratory Manual, Cold Spring Harbor, N.Y., 1989, and Ausubel et al., Current Protocols in Molecular Biology, Greene Publ. Assoc. and Wiley-Intersciences, 1992) or other nucleic acid amplification technologies well known in the art, for example, the ligase chain reaction (LCR) (see Wu and Wallace, Genomics 4:560 (1989), and Landegren et al., Science 241:1077 (1988)), transcription amplification (see Kwoh et al., Proc. Natl. Acad. Sci. USA 86:1173 (1989)), self-sustained sequence replication (see Guatelli et al., Proc. Nat. Acad. Sci. USA 87:1874 (1990)) and nucleic acid based sequence amplification (NASBA) (J. Compton, Nucleic-Acid Based Sequence Amplification; Nature 350 (6313): 91, Mar. 7, 1991).

In some embodiments, a majority or all target nucleic acids in a sample may be amplified. In some embodiments, a desired subpopulation of target nucleic acids may be amplified. For example, in a wastewater sample suspected of or known to comprise bacteria, mycobacteria and fungi, it may be desired to preferentially amplify bacterial target nucleic acids prior to use in the disclosed methods. In some embodiments, selected regions of target nucleic acids may be amplified, such as for example rRNA, such as 16S rRNA.

In some embodiments, a plurality of different target nucleic acids may be detected, identified and/or quantified in a sample. Plurality as used herein means at least two. In the context of an environmental sample, such as soil, water, wastewater, mud, vegetal extract, wood, biological material, marine or estuarine sediment, industrial effluents, gas, mineral extracts, sand, natural excrements, meteorites etc., which may contain a mixture of different organisms, a plurality of different target nucleic acids may be detected, identified and/or quantified in the sample, for example, about 2, about 3, about 4, about 5, about 10, about 15, or about 20 target nucleic acids may be detected, identified and/or quantified.

The target nucleic acids may be detected, identified and/or quantified using multiple nucleotide primers in one or more series of such nucleotide primers.

The primers for use in the disclosed methods and kits are typically oligonucleotides of, generally, 15 to 120 bases in length. Such primers can be prepared by any suitable method, including, for example, direct chemical synthesis or cloning and restriction of appropriate sequences. Not all bases in the primer need reflect the sequence of the template molecule to which the primer will bind; the primer need only contain sufficient complementary bases to enable the primer to bind to the target nucleic acid template. The primer may include additional bases at the 5′ end, for example in the form of a poly dA or poly dT tail to vary the length of the primers within a series of primers as described herein.

Methods for preparing and using primers are described in, for example, Sambrook, J. and D. W. Russell (2001; Molecular Cloning: A Laboratory Manual, 3rd ea., vol. 1-3, Cold Spring Harbor Press, Cold Spring Harbor N.Y.), Ausubel, F. M. et al. (1999; Short Protocols in Molecular Biology, 4d ea., John Wiley & Sons, New York N.Y.), and Innis, M. et al. (1990; PCR Protocols. A Guide to Methods and Applications, Academic Press, San Diego Calif.). The primers may be derived from a known sequence, for example, by using computer programs intended for that purpose such as Primer (Version 0.5, 1991, Whitehead Institute for Biomedical Research, Cambridge Mass.).

Oligonucleotides for use as primers may be selected using software known in the art for such purpose. For example, OLIGO 4.06 primer analysis software (available from National Biosciences, Plymouth, Minn.) is useful for the selection of primers of up to 30-100 nucleotides each, and for the analysis of larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome 5 Research, Cambridge Mass.) allows the user to input a “mispriming library,” in which sequences to avoid as primer binding sites are user-specified. Primer3 is useful, in particular, for the selection of nucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that bind or hybridize to either the most conserved or least conserved regions of aligned nucleic acid sequences. Hence, this program is useful for identification of both unique and conserved nucleotides and polynucleotide fragments.

A plurality of primers may form a series of primers. At least one series of primers is used in the disclosed methods. Each series of primers comprises between about 3 and about 20 primers. For example, a series may comprise about 3 to about 5 primers, about 5 to about 10 primers, about 8 to about 12 primers, about 10 to about 15 primers, about 13 to about 18 primers, or about 15 to about 20 primers. For example, a series may comprise about 3, 4, 5, 6, 7, 8, 9, 10, 11, 12, 13, 14, 15, 16, 17, 18, 19 or 20 primers.

Each primer may be present in one or more copy numbers within a series.

Different primers within a series may be distinguished during analysis or measurement by way of different primer lengths or different labels, or both. Where the primers within a series are distinguished on the basis of their length, at least one primer within the series may have a different length to the other members of the series. Alternatively, each primer within a series may have a different length to each other member of the series. Where the differences in the length of the primers in a series is intended for the purpose of discriminating between the primers, the differences must be sufficient to enable the distinguishing of each of the different primers. The skilled addressee will recognize that the method of analysis of the primers will influence the degree of difference required between primers, with some methods of analysis capable of discriminating differences of about 1, 2, 3, 4 or 5 bases (such as capillary electrophoresis and polyacrylamide gel electrophoresis) and others capable of discriminating differences of greater than about 5, 6, 7 or 8 bases (such as agarose gel electrophoresis). Generally, the differences may range from about 2 to about 70 nucleotides. Typically, the differences may be about 5 to about 10 nucleotides, about 11 to about 15 nucleotides, about 16 to about 20, about 21 to about 30 nucleotides, about 31 to about 40 nucleotides, about 41 to about 50 nucleotides, about 51 to about 60 nucleotides, or about 61 to about 70 nucleotides.

In some embodiments, the different lengths may be obtained, by way of including a nucleic acid tail of different lengths into one or more of, or each of, the primers within a series. Nucleic acid tails that may be used are well known in the art and include, but are not limited to, poly dA tails and poly dT tails. The length of a nucleic acid tail for individual members of a series of primers may range from about 2 to about 50 nucleotides. Typically, the length of a nucleic acid tail may be about to about 10 nucleotides, about 11 to about 15 nucleotides, about 15 to about 20 nucleotides, about 21 to about 25 nucleotides, about 25 to about 30 nucleotides, about 31 to about 35 nucleotides, about 35 to about 40 nucleotides, about 41 to about 45 nucleotides, or about 45 to about 50 nucleotides. In some embodiments, the length of a nucleic acid tail is about 4, about 8, about 12, about 18, about 24 or about 30 nucleotides.

Where one or more of the primers within a series are distinguished on the basis of their different labels, at least one primer within the series may have a different label to the other members of the series. Alternatively, each primer within a series may have a different label to each other member of the series. Each label is capable of emitting a detectable signal that distinguishes the different primers within the series. Labels that may be used in the disclosed methods are well known in the art and include, but are not limited to, those emitting a fluorescent signal, a chemiluminescent signal, a luminescent signal, a radioactive signal or a biotinylated signal. Examples of labels emitting fluorescent signals include fluorescein, rhodamine, coumarin, cyanine, fluorescent nanoparticles and fluorescent quantum dyes. Specific examples of fluorescent dyes include 3-(ε-carboxypentyl)-3′-ethyl-5,5′-dimethyloxa-carbocyanine (CYA), 6-carboxy fluorescein (FAM), 5&6-carboxyrhodamine-110 (R110), 6-carboxyrhodamine-6G (R6G), N′,N′,N′,N′-tetramethyl-6-carboxyrhodamine (TAMRA), 6-carboxy-X-rhodamine (ROX), 2′,4′,5′,7′,-tetrachloro-4-7-dichlorofluorescein (TET) and 2′,7′-dimethoxy-4′,5′-6 carbdxyrhodamine (JOE). Examples of labels emitting chemiluminiscent signals include 1,2-dioxetane, cyalume, oxalyl chloride, tetrakis(dimethylamino)ethylene, pyrogallol, lucigenin and luminol while those emitting radioactive signals include gamma-radioactive isotopes of iodine.

The label may be directly or indirectly coupled to the primer as described above. In one embodiment, the label may be coupled to the primer after binding of the primer to its target nucleic acid by a single chain primer extension reaction which comprises the extension of the bound primer with a single nucleotide labeled with a detectable signal. Single nucleotides that may be used for this purpose are well known in the art and include, but are not limited to, the dideoxynucleoside triphosphates (ddNTPs) ddATP, ddTTP, ddCTP and ddGTP. Typically, a fluorescently labeled dideoxynucleoside triphosphate (ddNTP) or a dye-terminator is added to the 3′ end of a primer upon its successful annealing to target nucleic acid. Due to the lack of a 3′-OH group on the added ddNTP to form a phosphodiester bond, the chain extension reaction is terminated. The label on the ddNTP is thus coupled to the nucleotide primer.

One member within each series of nucleotide primers has a lower level of specificity than other members of the series. In one embodiment, each member of a series, other than the one member with the lower level of specificity, is specific for a different target nucleic acid. In other embodiments, at least one nucleotide primer within a series is capable of binding to multiple target nucleic acids. In other embodiments, a first plurality of members of a series are each capable of binding to multiple target nucleic acids and a second plurality of members of the series is each specific for a different target.

In one embodiment, the nucleotide primers within a series have a hierarchical level of specificity. For example, a series may comprise a primer at a higher hierarchical level and a plurality of primers at a lower hierarchical level. A primer for a higher hierarchy level has a lower level of specificity in that the primer is capable of binding to more target nucleic acids than a primer for a lower hierarchy level which has a higher level of specificity. A primer for the highest-ranking hierarchy level within a series may, for example, bind all of the target nucleic acids in a sample.

In one embodiment, a series may comprise a plurality of species-specific primers and a non-species-specific primer. For example, a series may comprise a domain-specific primer and a plurality of primers selected from the group consisting of phylum-specific primers, class-specific primers, order-specific primers, family-specific primers, genus-specific primers and species-specific primers, or may comprise a phylum-specific primer and a plurality of primers selected from the group consisting of class-specific primers, order-specific primers, family-specific primers, genus-specific primers and species-specific primers, or may comprise a class-specific primer and a plurality of primers selected from the group consisting of order-specific primers, family-specific primers, genus-specific primers and species-specific primers, or may comprise an order-specific primer and a plurality of primers selected from the group consisting of family-specific primers, genus-specific primers and species-specific primers, or may comprise a family-specific primer and a plurality of primers selected from the group consisting of genus-specific primers and species-specific primers, or may comprise a genus-specific primer and a plurality of species-specific primers. It is expected that a primer with a domain-level specificity is capable of binding more target nucleic acids than a primer with a lower hierarchical specificity. A domain as used in this context means the highest rank of organisms in a taxonomic hierarchy. Typically, a taxonomic hierarchy ranks from a domain, followed by a kingdom, a phylum, a class, an order, a family, a genus and a species. Each hierarchical rank may further include a prefix, for example, a prefix sub- indicates a rank below, a prefix super- indicates a rank above, and the prefix infra- indicates a rank below sub-. For example, a super-domain is higher ranking than a domain, which is higher ranking than a sub-domain, which is higher ranking than an infra-domain. The prefixes for a kingdom, a phylum, a class, an order, a family, a genus and a species shall be construed accordingly.

As described above, multiple series of nucleotide primers may be used in the disclosed methods. For example, two, three, four, five, six, seven, eight, nine, ten, eleven, twelve, thirteen, fourteen or fifteen series may be used to analyze a sample. Where more than one multiplex reaction is conducted, several series of nucleotide primers may be used. For example, series ‘A’ may be used in a four-plex reaction, series ‘B’ in a six-plex reaction and series ‘C’ in a seven-plex reaction. The separate multiplex reactions may also be combined in various permutations to further increase the multiplicity of a multiplex reaction. For example, series ‘B’ in a six-plex reaction may be combined with series ‘C’ in a seven-plex reaction to increase the multiplicity to a ten-plex reaction.

Where two series are used, a first aliquot of a sample is contacted with a first series of nucleotide primers and a second aliquot of the sample is separately contacted with a second series of nucleotide primers, the first and second series having at least one nucleotide primer in common.

Similarly, where three series are used, a first aliquot of a sample is contacted with a first series of nucleotide primers, a second aliquot of the sample is separately contacted, with a second series of nucleotide primers, and a third aliquot of the sample is separately contacted with a third series of nucleotide primers. The first and second series may have at least one nucleotide primer in common, the second and third series may have at least one nucleotide primer in common, and the first and third series may have at least one nucleotide primer in common. For the avoidance of confusion, the first, second and third series may all have at least one nucleotide primer in common.

In one embodiment, the method is a method for determining constituent organisms in a heterogenous sample. The constituent organisms may be prokaryotes, such as bacteria (for example, gram positive bacteria, green filamentous bacteria, spirochetes, proteobacteria, cyanobacteria, planctomyces, bacteroides cytophaga, thermotoga, aquifex) and archae (for example, halophiles, methanosarcina, methanobacterium, methanococcus, thermoproteus, pyrodicticum), or eukaryotes, such as fungi (for example, phycomycetes, ascomycetes and basidiomycetes), yeast (for example, saccharomyces, kluyveromyces), and plants (for example, algae, lichens etc.).

The conditions allowing the primers to bind target nucleic acids are well known in the art, or may be easily determined using common experimental protocols. In each case, suitable protocols and reagents will largely depend on individual circumstances. Guidance may be obtained from a variety of sources, such as for example Sambrook et al., Molecular Cloning: A Laboratory Manual, Cold Spring Harbor, N.Y., 1989, and Ausubel et al., Current Protocols in Molecular Biology, Greene Publ. Assoc. and Wiley-Intersciences, 1992. A person skilled in the art would readily appreciate that various parameters of these procedures may be altered without affecting the ability to achieve the desired result. For example, the amount of target nucleic acids used may be varied depending on the amount of material or sample available, such as RNA or DNA, or the optimal amount of target nucleic acids required for efficient binding.

Typically, conditions suitable for binding of the primers to the target nucleic acids include salt concentrations of less than about 1M, more usually less than about 500 mM and less than about 200 mM. Suitable temperatures can be as low as 5° C., but are typically greater than 22° C., more typically greater than about 30° C., and preferably in excess of about 37° C. Binding of primers to target nucleic acids is preferably performed under stringent conditions. Stringent conditions are sequence-dependent and are different under different circumstances. Longer primers may require higher temperatures for specific binding. As other factors may affect the stringency of binding, including base composition and length of the complementary strands, presence of organic solvents and extent of base mismatching, the combination of parameters is more important than the absolute measure of any one alone. Generally, stringent conditions are selected to be about 5° C. lower than the thermal melting point (Tm) for the specific sequence at a defined ionic strength and pH. The Tm is the temperature (under defined ionic strength, pH and nucleic acid composition) at which 50% of the primers complementary to the target nucleic acid bind to the target nucleic acid at equilibrium. Typically, stringent conditions include a salt concentration of at least 0.01 M to no more than 1 M Na ion concentration (or other salts) at a pH 7.0 to 8.3 and a temperature of at least 25° C.

While binding of the primers to the nucleic acid targets are preferably conducted under conditions of high stringency as described above, binding may also be conducted under conditions of medium stringency or low stringency. Low stringency conditions may correspond to binding performed at 50° C. in 2×SSC.

There are numerous conditions and factors, well known to those skilled in the art, which may be employed to alter the stringency of such binding. For instance, the length and nature (DNA, RNA, base composition) of the primer to be bound to a target nucleic acid; concentration of salts and other components, such as the presence or absence of formamide, dextran sulfate, and polyethylene glycol; and altering the temperature of the binding and/or washing steps.

As discussed herein, the bound primers may be labeled by way of extending the primer with a single labeled nucleotide. The single nucleotide chain extension typically requires single-stranded DNA template (i.e the target nucleic acids), a nucleotide primer, an enzyme (i.e a DNA polymerase), labeled nucleotides, and modified nucleotides that terminate DNA strand elongation (for example, dideoxynucleotides or ddNTPs). These dideoxynucleotides are the chain-terminating nucleotides lacking a 3′-OH group required for the formation of a phosphodiester bond between two nucleotides during DNA strand elongation. Incorporation of a dideoxynucleotide into the nascent (elongating) DNA strand therefore terminates DNA strand extension. The labeled primers are heat denatured, and may be separated by size (with a resolution of just one nucleotide) as discussed further below.

Suitable conditions for extending the primer with a single labeled nucleotide are essentially the same as those applied in the extension/elongation step of a PCR. The DNA polymerase (for example, the Taq polymerase) adds a ddNTP to the primer at a suitable temperature that is typically dependent on the DNA polymerase used. Where Taq polymerase is used, for example, an optimum temperature of 70-74° C. may be used. Typically, a temperature of about 72° C. is used. The skilled addressee will recognize that any suitable enzyme for extending the primer may be used, additional examples including DNA Polymerase I, DNA Polymerase II, DNA Polymerase III holoenzyme, DNA Polymerase IV (DinB), Terminal Deoxynucleotidyl Transferase, RNA Polymerase I, RNA Polymerase II, RNA Polymerase III, T7 RNA Polymerase, and Reverse Transcriptase.

Multiple cycles of the conditions for binding of the primers to the target nucleic acids and extension of the primers with at least one single nucleotide labeled with a detectable signal may be conducted. For example, 15 to 50 cycles may be conducted. Typically, about 20, about 25, about 30 or about 35 cycles are conducted.

The products (i.e. the extended labeled primers) may be purified prior to analysis, for example to remove unincorporated labeled single nucleotides remaining in the reaction mixture. Purification protocols are well known to those skilled in the art and include, but are not limited to, various forms of chromatographic separation (for example, high performance liquid chromatography), enzyme-based digestion (for example, using alkaline phosphatase), spin column purification, or electrophoretic separation.

The reaction products may be separated according to their different sizes and/or different labels prior to or concurrently with detection and measurement of the detectable signals emitted by the labels coupled to the primers. Separation techniques that may be used include electrophoresis (for example, gel micro-channel and capillary electrophoresis), chromatography (for example, high performance liquid chromatography), and mass spectrometry (for example, MALDI-TOF).

The reaction products may be analyzed using a DNA autosequencer equipped with detectors, such as fluorescence, chemiluminescence, luminescence or radioactive detectors.

The lengths and concentrations of the extended primers may be calculated according to internal standards, for example internal standards consisting of different fluorescently labeled oligonucleotides varying from 13-88 by in length and 100-1000 pM in concentration. By knowing the extension efficiency of individual primers, the relative intensity of labeled primers that are more specific to labeled primers that are less specific may be determined, and if desired, may be used to calculate the relative quantity of different target nucleic acids in a sample.

The detectable signal of a labeled primer may be quantified by application of a calibration factor of one or more target nucleic acids. The calibration factor may be obtained by determining the ratio of the intensity of a detectable signal of a labeled or extended primer having a higher level of specificity relative to the intensity of a detectable signal of a labeled or extended primer having a lower level of specificity. The following equations may be used in determining the relative abundance of a target nucleic acid.

The concentration of a specific extended primer may be quantified using the following equation:

$$\frac{Peak area of extended primer}{( {C}_{p} ) ( volume of injected sample )} = \frac{ \begin{matrix}Peak area of associated \\ std oligonucleotide\end{matrix} }{( {C}_{std} ) ( volume of std )}$$

where Cₚ represents the molar concentration of a specific primer extended in the HOPE reactions for the sample; C_{std} represents the molar concentration of the associated standard oligonucleotide.

Calibration factors for each primer may be obtained using the associated standard oligonucleotide as template, and determined using the following equation:

$$C {F}_{A - B} = \frac{Concentration of extended primer A , {C}_{a}}{Concentration of extended primer B , {C}_{b}}$$

where primer B has a lower level of specificity compared to primer A.

The relative abundance of nucleic acids targeted by primer A with respect to those targeted by primer B can then be calculated as follow:

$$Relative abundance of target ( \% ) = \frac{{C}_{a}}{{C}_{b} \times C {F}_{A - B}} \times 100 \%$$

One embodiment of the method described herein is illustrated in FIG. 1. The initial step of this embodiment of the disclosed method involves the strand extension reaction used in ‘minisequencing’ to extend a single fluorescently labeled dideoxynucleoside triphosphate (ddATP, ddTTP, ddGTP or ddCTP) or a dye-terminator at the 3′-end of a primer upon its successful annealing to the targeted region of purified PCR-amplified rRNA genes. In this step, multiple oligonucleotide primers, which are designed to target sequences at different levels of specificities (i.e. domain, phylum, etc., species), and modified with different lengths of poly dA tails at the 5′-end of individual primers, are added. After single base extension, the HOPE products are purified and analyzed using a DNA autosequencer equipped with four-color detectors. Due to differences in length and the dye-terminator type/color added, these extended hierarchical primers can be separated and identified, and their lengths and concentrations calculated according to the internal standards consisting of different fluorescently labeled oligonucleotides. As a primer with domain-level specificity can prime onto more DNA templates than a group-specific primer, by knowing the extension efficiency of individual primers, the relative intensity of a specific labeled primer to a broad specific primer can be obtained, and this ratio will represent the relative abundance of the targeting rRNA fragment in a PCR mixture. HOPE procedure can be completed within 90 min, hence enabling rapid identification of multiple microbial targets in environmental samples.

Kits for use in the disclosed methods are also provided. The kit may comprise at least one series of nucleotide primers capable of binding to a plurality of target nucleic acids, wherein one member within each series has a lower level of specificity than other members of the series; and instructions for use in accordance with the disclosed methods. In some embodiments, the kit may include multiple series of primers, for example two, three, four, five, six, seven, eight, nine, ten, eleven, twelve, thirteen, fourteen or fifteen series. A series of primers may comprise between about 3 to about 20 nucleotide primers as described herein.

In some embodiments, the kit may further comprise a set of labels having a detectable signal capable of binding to the nucleotide primers. Exemplary labels include labels emitting fluorescent signals, chemiluminiscent signals, luminescent signals, radioactive signals, and biotinylated signals as described herein.

In some embodiments, the kit may include nucleic acid tails comprising between about 2 to about 70 nucleotides. Exemplary nucleic acid tails that may be included in the kit are poly dA tails and poly dT tails as described herein.

In some embodiments, the kit may include additional reagents such as reagents for sample preparation, sample amplification, single nucleotide chain extension of primers, and purification of reaction products. Reagents for sample preparation include, but are not limited to, buffers for suspension or dilution of a sample, and alcohols (such as phenol, chloroform, isopropanol and ethanol) and salt solutions (such as sodium or ammonium acetate) for extraction of nucleic acids.

Reagents for sample amplification include, but are not limited to, an enzyme suitable for extending the primer (such as Taq polymerase or any other enzymes as described herein), deoxynucleotide triphosphates (such as dATP, dTTP, dCTP and dGTP), buffer solutions (such as potassium chloride and Tris-HCl), and divalent cations (such as magnesium chloride).

Reagents for single nucleotide chain extension of primers include, but are not limited to, an enzyme suitable for extending the primer (such as Taq polymerase or any other enzymes as described herein), dideoxynucleoside triphosphates (ddNTPs) (such as ddATP, ddTTP, ddCTP and ddGTP as described herein), suitable buffer solutions (such as potassium chloride and Tris-HCl), and divalent cations (such as magnesium chloride). The ddNTPs may optionally be coupled to a label as described herein.

Reagents for purification of reaction products include, but are not limited to, reagents for chromatographic separation (such as elution buffers, such as TE buffer and Tris-HCl buffers), enzyme-based digestion (such as alkaline phosphatase), spin column purification (such as elutants imidazole, ammonium chloride, histidine etc.), chemical precipitation (such as phenol, isopropanol, sodium acetate, Tris-EDTA, etc.), and electrophoresis separation (such as agarose gel, polyacrylamide, sodium dodecyl sulfate, sodium dodecyl phosphate etc.).

In some embodiments, the kit may include internal standards, such as fluorescently labeled oligonucleotides, to facilitate quantification of target nucleic acids.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a schematic diagram illustrating the concept of a hierarchical oligonucleotide primer extension (HOPE) approach for multiplex identification and quantitative analysis.

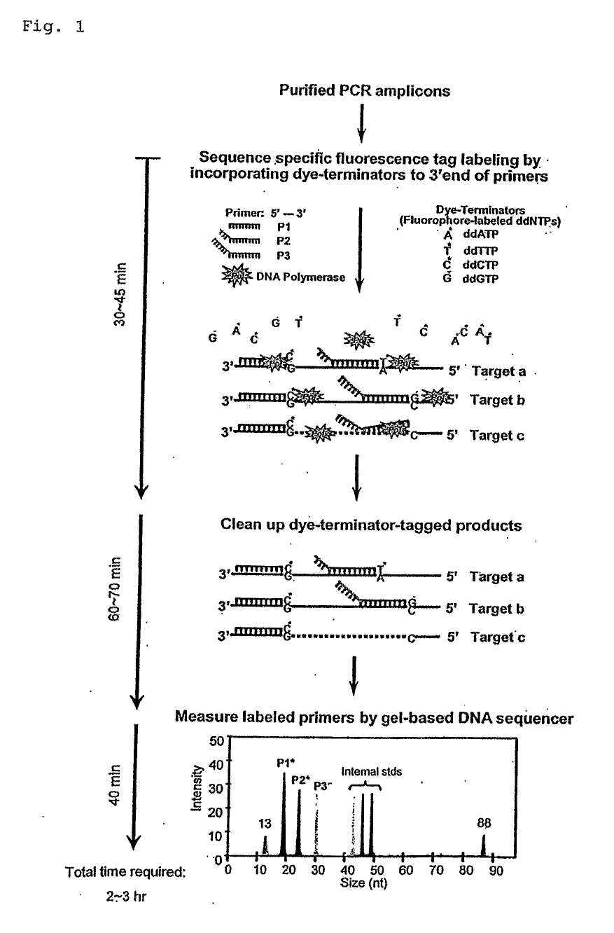

FIG. 2 shows the sensitivity of DNA sequencers for oligonucleotide separation and fluorescence measurement. (a) Sequences of synthetic oligonucleotides with a Cy5 modification at the 5′ end. (b) A size-based electrophoregram of the four Cy5-labeled oligonucleotides in different concentrations separated by capillary electrophoresis. (c) Linearity in the concentrations (94˜1857 pM) of Cy5-labeled oligonucleotides and fluorescence intensities in terms of peak areas. (d) Linearity in the concentrations (2.4˜46.4 pM) of Cy5-labeled oligonucleotides and fluorescence intensities in terms of peak areas.

FIG. 3 shows the effect of poly dA tail length on the efficiency of primer extension with dye-terminators. (a) Sequences for selected regions of E. coli 16S rRNA gene and for oligonucleotide primers (EUB338) modified with different lengths of poly dA tails. Nucleotides identical to those found in the EUB338 primer without the poly dA tail are represented by hyphens. (b) Decrease in primer extension efficiency as affected by length of poly dA tail added to the 5′ end of the EUB338 primer. The extension efficiencies presented as percentages were calculated based on the ratio of the concentration of each ddCTP-coupled primer to the concentration of each of the ddCTP-coupled primer EUB338 without the poly dA tail. The data points represent the average values derived from two reactions and repeated capillary electrophoresis analysis. The error bars represent standard deviation for the average data points.

FIG. 4 Phylogenetic tree of Bacteroides spp. and the specificity of primers used in multiplexing HOPE. The dendrogram is constructed with neighbor-joining method using ARB and rooted with Escherichia coli. The scale bar corresponds to 10 nucleotide substitutions per 100 nucleotide positions. The primers used in the 4-plexing (open circle), 6-plexing (filled circle) and 7-plexing (filled square) reactions are indicated at the right of the tree. The primer specificity is specified by the position of the primers or the bracket. The type of the extended nucleotide is indicated within brackets after the primer name. The terminal restriction fragment (tRF) lengths of Bacteroides spp. that are in silico analyzed with primers Bac32F and Bac708R and restriction enzyme AciI using the tRFcut add-in program in ARB are indicated in brackets after the species names.

FIG. 5 shows the effect of annealing temperature on the efficiency of multiplex HOPE. The reactions were conducted using B. thetaiotaomicron 16S rRNA gene amplicons and a primer mixture containing (a) EUB338Ia, (b) BAC303-5a, (c) BTH274-15a, and (d) BTH584-16a. Twenty-five cycles of thermal cycling (denaturation at 96° C. for 10 sec, annealing at the evaluated temperatures for 30 sec, and extension at 72° C. for 15 sec) were applied. For each evaluated temperature, the corresponding efficiency was calculated as a percentage of the maximum encountered for that primer. The melting temperature (Tm) for each primer was calculated according to the formula: 2(A+T)+4(G+C) without the dA.

FIG. 6 shows the effect of cycle number on multiplex HOPE with dye-terminators. The reactions were carried out using the thermal cycling conditions: denaturation at 96° C. for 30 sec, annealing at 60° C. for 30 sec and extension at 72° C. for 30 sec. The experiments were conducted using B. thetaiotaomicron 16S rRNA gene amplicons and a primer mixture containing EUB338Ia, BAC303-5a, BTH274-15a, and BTH584-16a. The data points represent the average values derived from two reactions and repeated capillary electrophoresis analyses. The error bars represent standard deviation for average data points.

FIG. 7 shows the distribution of conversion factors for the primers BAC303-5a (), BTH274-15a (◯), and BTH584-16a (▾) against the universal primer EUB338Ia. The primer-to-target ratio (molar basis) ranged from 250 to 16000. The experiment was conducted using fixed primer concentrations, and varying target concentrations as indicated in Example 1 below. The data points represent the average values derived from three reactions. The error bars represent standard deviation for average data points.

FIG. 8 shows the specificity of the multiplex HOPE. The individual Bacteroides spp. template is used in a 4-plexing HOPE reaction where there is inclusive of primers EUB338, BAC303-5a, BTH274-15a and BTH584-16a. (a) Sequences of the primers and of the binding regions of selected reference targets. For each primer, the complementary sequence for the first bacterial species is shown. When identical sequence compositions were encountered in the subsequent species, they are represented by double dashes. For the primer EUB338, the nucleotides that were underlined indicate the sequence for the primer EUB338Ia. (b) Specificity of multiplex HOPE with dye-terminators. The individual Bacteroides species template was indicated at the top right of each size-based electrophoregrams. Dash and solid peaks represent D4-ddUTP- and D2-ddCTP-coupled primers, respectively.

FIG. 9 shows the sensitivity of HOPE with dye-terminators. Two primers, (a) BTH274-15A and (b) BTH584-16A, were used together with a serial concentration of 16S rRNA gene amplicons of B. thetaiotaomicron from 1 pg to 5000 pg. The reactions were carried out in a total volume of 20 μl (open circle), 10 μl (open square) and 5 μl (open inverted triangle) using B. thetaiotaomicron amplicons alone. 5 μl () reacting with B. thetaiotaomicron, plus 100 ng of L. acidophilus amplicons were also performed. Forty cycles of thermal cycling conditions (denaturation at 96° C. for 10 sec, annealing at 60° C. for 30 sec, and extension at 72° C. for 15 sec) were applied. The data points represent the average values derived from one reaction with repeated analysis of capillary electrophoresis. The error bars represent the standard deviation for average data points.

FIG. 10 shows the various formats of nucleic acid, including genomic DNA (a) and native RNA (b) that can be analyzed in the multiplex HOPE method.

FIG. 11 The electrophoregrams of 6-plexing reaction products generated from mixed 16S rRNA gene amplicons of Bacteroides tectus, Bacteroides fragilis and Bacteroides thetaiotaomicron (a); 7-plexing reaction products generated from mixed 16S rRNA gene amplicons of Bacteroides intestinalis, B. fragilis, Bacteroides acidifaciens, Bacteroides uniformis and Bacteroides pyogenes (b); and 7-plexing reaction products generated from 16S rRNA gene amplicons of wastewater influent (c). The dashed line, solid line and solid peak represent primers extended with D4-ddUTP-, D3-ddGTP and D2-ddCTP, respectively. The primers detected and internal standards (GT40-D4 and GT46-D2) are labeled beside the corresponding peaks in panel (c).

FIG. 12 shows another embodiment of the HOPE reaction. PCR amplicons, fluorophore-labeled ddNTPs, DNA polymerase and oligonucleotide primers are mixed together to form a multiplex HOPE reaction mixture. The mixture undergoes 20-25 cycles of denaturation (96° C.), annealing (60-64° C.) and single-base extension (72° C.). An appropriate amount of the product mixture is then mixed with synthetic oligonucleotide standards, and subjected to capillary electrophoresis.

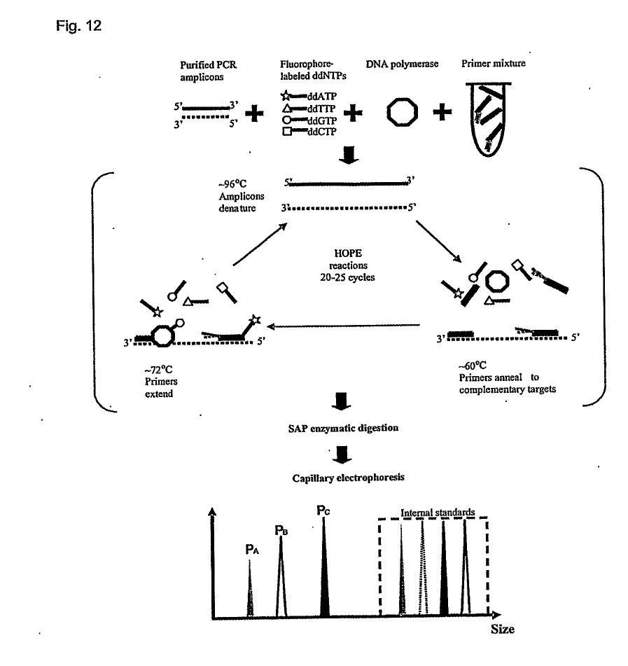

FIG. 13 shows the phylogenetic tree of Bacteroides spp. and the specificity of primers used in multiplex HOPE. Multiplex HOPE reaction one to five contains species-specific primers and a higher ranked primer specific to the B. fragilis cluster (Bfrg602_{—}19dA or Bth274[T]_{—}15dA). Multiplex HOPE reaction six contains primers targeting at sub-cluster (Bth274_{—}15dA), cluster (Bfrg602_{—}19dA), order (Bac303_{—}5dA) and domain (U1390_{—}17dA) levels.

FIG. 14 shows the capillary electrophoregrams for multiplex HOPE reactions one to six. For each sample, 30 ng of PCR amplicons and 2.5 μl of SNP premix were added to multiplex HOPE reaction one to six. As illustrated in the capillary electrophoregrams, Bacteroides spp. was detected at the domain-, order- and species-level for human feces H3. Black, red and green peaks represent primers that extended with dTAMRA-ddCTP, dROX-ddTTP, dR6G-ddATP, respectively. Internal standards were labeled and shown beside the corresponding peaks.

FIG. 15 shows the PCR amplification of microbial targets at various numbers of cycles to determine the optimal number of cycles.

### BEST MODE

Non-limiting examples of the invention will be further described in greater detail by reference to specific Examples, which should not be construed as in any way limiting the scope of the invention.

### Example 1

### Analysis of Microbial Targets in Environmental Samples

### (a) Materials and Methods

Influent and effluent samples of a local wastewater treatment system were taken, and total suspended solid including microbial cells were concentrated by centrifugation.

Total DNA from individual reference strains and samples of the local sewage treatment plant was extracted, and used as DNA template for the PCR amplification of 16S rRNA genes. The PCR (100 ml) contained 1× buffer solution (Promega), 2.0 mM of MgCl₂, 200 nM of each primer [11F, GTT TGA TCC TGG CTC AG and 1492R, GG(C/T) TAC CTT GTT ACG ACT T], 200 mM of each dNTP (dATP, dTTP, dGTP, dCTP), 0.5 U of Taq DNA polymerase (Promega) and 50-100 ng of genomic DNA. PCR amplification was carried out using Bio-Rad iCycler (Hercules, Calif.) under the following thermal program: initial denaturation (95° C., 3 min), 30 cycles of 95° C. (30 s), 55° C. (30 s) and 72° C. (30 s), and final extension (72° C., 5 min). After verifying amplification by agarose gel electrophoresis, the PCR products were purified using QIAquick PCR purification kit (Qiagen) according to the manufacturer's instructions. The concentrations of purified PCR products were quantified by UV absorbance measurement using a DU 800 spectrophometer (Beckman Coulter, Fullerton, Calif.).

Purified PCR-amplified products were used in the HOPE reaction. The HOPE reaction, carried out in a total volume of 5 to 20 μl, contained 5-10 pico-mole (pmole) of individual oligonucleotide primers, 10-20 ng of purified templates, and 1× pre-mixed solution of the CEQ™ SNP-Primer Extension Kit (Beckman Coulter, Fullerton, Calif.). The premix aliquot (2×) containing DNA polymerase (9%, v/v), reaction buffer (18.2%, v/v), and fluorescently labeled dideoxynucleotides (ddUTP, ddGTP, ddATP, and ddCTP, 18.2% (v/v)) provided in the kit was prepared in accordance with the manufacturer's recommendation. The ddNTPs or dye-terminators were labeled with four different WellRed fluorescent dyes (D1, D2, D3 and D4) (Beckman Coulter). The oligonucleotide primers used were synthesized and purified using HPLC. The primer extension reaction was carried out using the Bio-Rad iCycler under the following thermal cycling program: 20 cycles of 96° C. (10 s), 60° C. (30 s) and 72° C. (15 s).

After the primer extension reaction, 1 U of shrimp alkaline phosphatase (Roche Applied Science, Penzberg, Germany) was added to the reaction mixture to hydrolyze 5′ phosphate groups of unincorporated dye-terminators. The mixture was incubated at 37° C. for 60 min, and the reaction was stopped by thermal denaturation at 85° C. for 10 min. Alternatively, to shorten reaction time, the HOPE reaction products can be purified using Microcon-YM3 spin column (Millipore). Table 1b lists all the primers used.

The HOPE reaction products were analyzed using a CEQ™ 8000 genetic analysis system with a four-color detection capability (Beckman Coulter). Prior to capillary electrophoresis, 1 μL of diluted HOPE reaction products was mixed with 1 μL of internal concentration and size standard (see below, 500-1000 pM), 0.2 μL of GenomeLab™ DNA size standard 80 kit (Beckman Coulter), and 39.8 μL of sample loading solution (Beckman Coulter). The mixture was transferred into a 96-well plate, and overlaid with one drop of mineral oil. The plate was then loaded into the CEQ™ 8000 system together with a buffer plate filled with separation buffer (Beckman Coulter). The electrophoresis program comprised a denaturation step (90° C., 120 s), an injection step under a voltage of 2.1 kV for 15 s (or 6 kV for 5 s), and a separation step (58° C., 16 min) under a voltage of 6.0 kV. In order to detect labeled oligonucleotides present in low concentrations, injection sample volume and injection time were increased up to 2 μL and 40 s, respectively. Fluorescence intensity data were automatically collected and subsequently analyzed by the software provided for CEQ™ 8000 system.

The electrophoretic sizes of individual oligonucleotides were determined and calibrated with the internal size standards using a default linear model and dye calibration parameters (SNP ver 1) built into the software. The internal concentration and size standards used contained a mixture of four different Cy5-labeled oligonucleotides at four different lengths (i.e. 50-Cy5-[GT]x-30, x=8, 13, 18 and 23), one D2-labeled oligonucleotide (50-D2-[GT]23-30), and one D4-labeled oligonucleotide (i.e. 50-D4-[GT]20-30) (Table 1b). They were synthesized and HPLC-purified by Operon Biotechnologies (Cologne, Germany) or Sigma-Proligo France SAS (Paris, France). The concentration and purity of those oligonucleotides were calculated according to the optical density of oligonucleotides and dyes measured at the maximum absorbance, and the extinction coefficients of dyes. The Genometab™ DNA size standard 80 kit contains WellRed D1-labeled fragments at 13 and 88 by in length.

The concentrations of the dye-terminator-labeled oligonucleotide primers were quantified according to Equation (1):

$$Cp = \frac{{C}_{di} \times {A}_{p} \times {V}_{dt}}{{A}_{dt} \times {V}_{p}} \qquad ( 1 )$$

where Cp represents the molar concentration of a oligonucleotide primer that is incorporated with a specific dye-terminator; C_{di} represents the molar concentration of labeled internal oligonucleotide standard with a dye identical to the labeled oligonucleotide primer; Aₚ represents the peak area for the oligonucleotide primer; A_{di} represents the peak area for the internal oligonucleotide standard; Vₚ represents the volume of HOPE reaction products loaded to the sample loading solution; and V_{di} represents the volume of internal standard loaded to the sample loading solution.

The relative quantity of a specific target nucleic acid sequence quantified by a specific primer to the total targets quantified by a universal primer can be calculated according to the following Equation (2):

$$Relative abundance of the target ( \% ) = \frac{Cp}{{C}_{338 Ia} \times C {F}_{p - 338 Ia}} \times 100 \% \qquad ( 2 )$$

where Cp represents the molar concentration of a specific primer extended in the HOPE reaction; C338Ia represents the molar concentration of universal primer EUB338Ia extended in the HOPE reaction; and CFp-338Ia is the calibration factor for primer extension efficiency of the specific primer with respect to EUB338Ia obtained using the reference strain.

### (b) Experimental Results

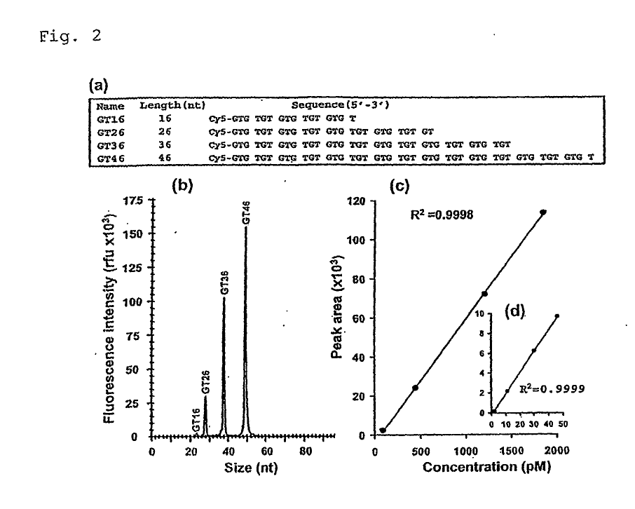

FIG. 2 shows the sensitivity of DNA sequencers for oligonucleotide separation and fluorescence measurement. A mixture of four different Cy5-labeled synthetic oligonucleotides (i.e., cy5-GT16, cy5-GT26, cy5-GT36 and cy5-GT46) at two different final concentration ranges, 2.4 to 46.4 pM and 94 to 1857 pM, were evaluated (FIG. 2a). Distinct and accurate length separation was obtained among those four Cy5-labeled oligonucleotides (FIG. 2b). The observed fluorescence intensity or peak areas were highly correlated (R²>0.9998) to the concentrations of those Cy5-labeled oligonucleotides (FIGS. 2c and 2d), with a dynamic range of at least 3 orders.

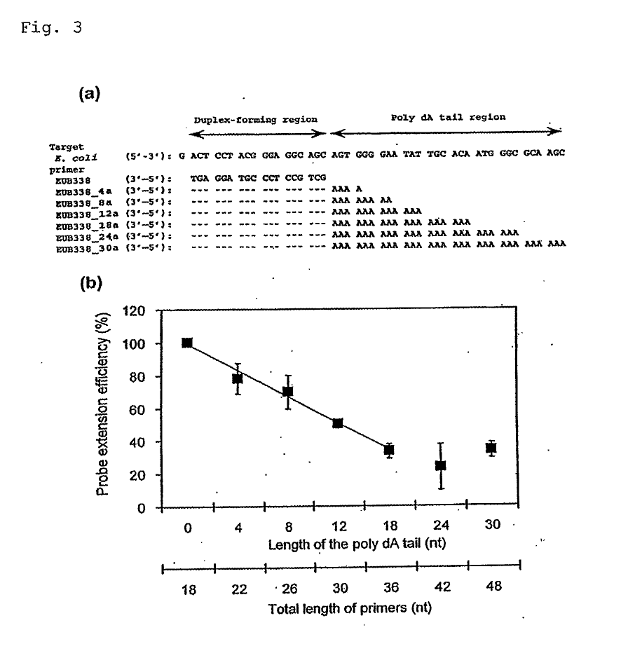

FIG. 3 shows the effects of poly dA length on single-base primer extension efficiency. Seven EUB338 primers modified with different lengths of poly dA tails from 0 to nucleotides (nt) were used in primer extension reactions (FIG. 3a and Table 1b). They were separately extended with a D2-labeled ddCTP. After analysis using a DNA autosequencer, the concentrations of individual extended primers were quantified, according to the observed fluorescent intensities using Equation (1). The relative primer extension efficiencies for those poly-dA attached EUB primers were calculated by assuming the extension efficiency of EUB338 as 100%. FIG. 3b shows that the efficiencies of single-base primer extension decreased linearly (3.6% per dA) with an increase in the length of poly dA tails up to 18 nt (R²=0.982). When the length of the poly dA tail exceeded 18 nt, the primer extension efficiencies fluctuated around 24-34%. A length of poly dA tails up to 18 nt was used hereafter.

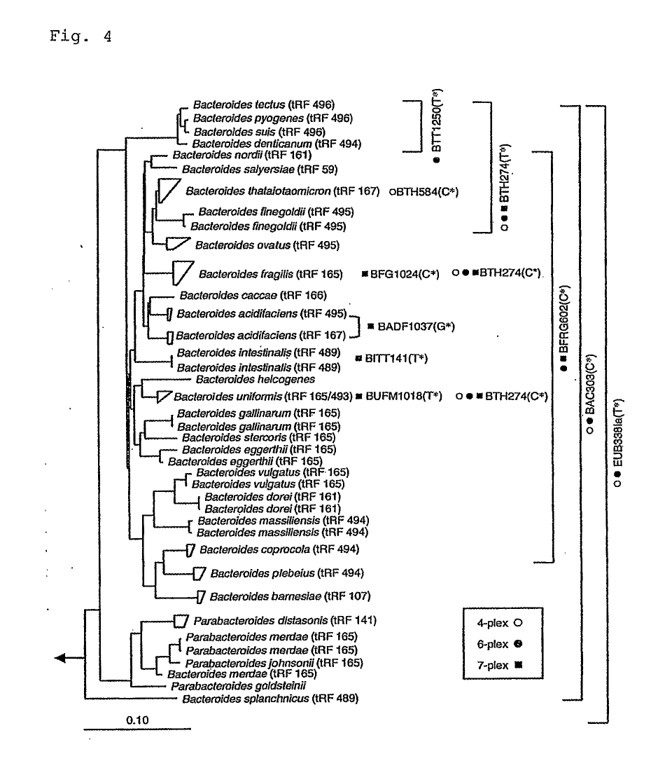

Initially, four different hierarchical oligonucleotide primers were used in a HOPE reaction. These four primers, namely EUB338Ia, BAC303-5a, BTH274-15a and BTH584-16a, were modified with 0, 5, 15 and 16 dAs, respectively, at the 5′-terminus. The specificities of these primers are indicated in Table 1b and FIG. 4.

The predicted extended nucleotide types of these four primers were analyzed in silico by the Match Probes function provided in the ARB using the ssu_jan04.arb database (www.arb-home.de) containing 28,289 nearly complete 16S rRNA sequences (41450 nt). The predicted type of nucleotide extended was mostly ddTTP (91.6%) for EUB338Ia, and ddCTP for BAC303-5a and BTH584-16a. For BTH274-15a, the extended nucleotide type was a ddTTP or a ddCTP for Bacteroides thetaiotaomicron or Bacteroides fragilis 16S rRNA gene, respectively. The specificities of other HOPE primers and their extended nucleotide types are indicated in FIG. 4.

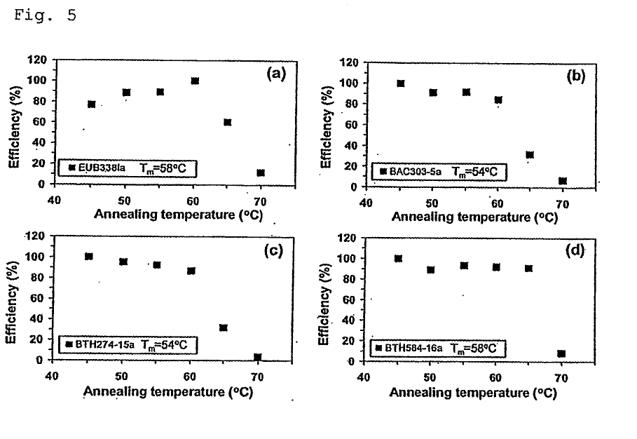

FIG. 5 shows the effects of the annealing temperature on the primer extension efficiency. Annealing temperature was varied from 45 to 70° C. at an increment of 5° C. After strand extension reactions, those labeled primers were quantified according to Equation (1). The relative primer extension efficiencies were normalized against the highest concentration observed for individual primers. The highest extension efficiencies for BAC303-5A, BTH274-15A and BTH584-16A occurred at an annealing temperature of 45° C., whereas that of EUB338Ia was 60° C. Between 45 to 60° C., all primers exhibited an extension efficiency between 80% and 100%. At a temperature higher than 65° C., the primer extension efficiencies for EUB338Ia, BAC303-5A and BTH274-15A rapidly decreased to less than 12.2%. For BTH584-16A, the primer extension efficiency decreased to 8.2% when the annealing temperature was higher than 70° C. To achieve a stringent condition for primer extension, an annealing temperature of 60° C. was used for subsequent experiments in this study.

Effects of duration time used in denaturation (10 s, and 60 s), annealing (5 s, 30 s and 60 s) and extension (15 s, 30 s and 60 s) on primer extension efficiency were investigated. The optimal duration obtained is 10 s for denaturation at 96° C., 30 s for annealing at 60° C. and 15 s for extension at 72° C.

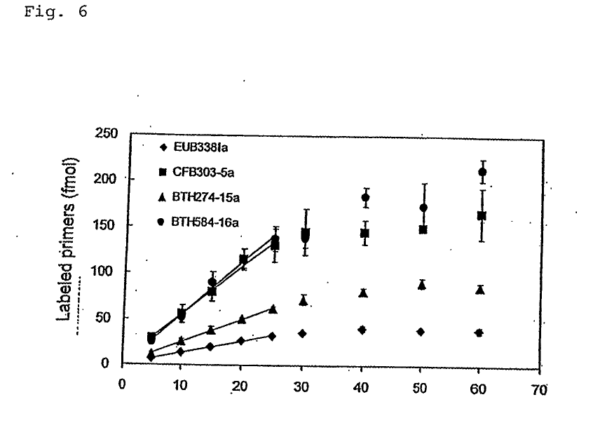

FIG. 6 shows the consistent results of primer extension. The results indicated that the amounts of extended primers increased linearly along with an increase in cycle number up to 25, and gradually reached a plateau after 30 cycles. The efficiency of primer extension remained constant with cycle numbers up to 25 cycles. Up to 25 cycles, the rates of increase for D2-ddCTP-labeled BTH584-16A and BAC303-5A were similar and higher than that of D4-ddUTP-labeled BTH274-15A and EUB338Ia. The slopes of primer extension for BTH584-16A, BAC303-5A, BTH274-15A and EUB338Ia were calculated to be 5.77, 5.23, 2.48 and 1.26 femto-moles per cycle (R²>0.99), respectively. In other words, the amounts of those BTH584-16A, BAC303-5A and BTH274-15A that have annealed onto the template and were successfully extended were 4.19, 4.15 and 1.92 times higher than the amount of EUB338Ia extended.

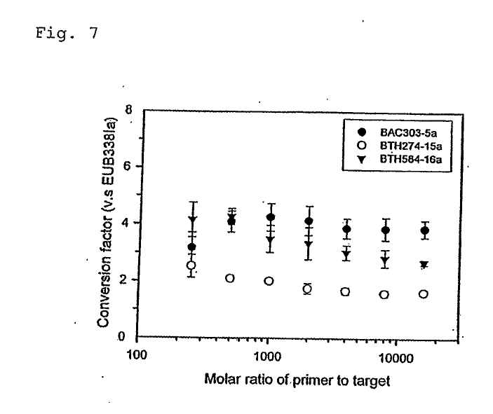

FIG. 7 shows the effects of primer-to-template ratio. For this study, the primers were fixed at 5 pmol for EUB338Ia and 10 pmol for BAC303-5A, BTH584-16A and BTH274-15A. A range of the primer-to-template ratio (based on EUB338Ia) from 250 to 16000 was used by varying the template quantity (i.e. B. thetaiotaomicron PCR amplicon) from 20 fmol to 0.3125 fmol. The amounts of primer extended from those four primers at individual primer-to-template ratios were normalized assuming the amount of the extended EUB338Ia as one. The normalized ratio between EUB338Ia and the D2-ddCTP-extended primers (BAC303-5A and BTH584-16A) were generally higher than that between EUB338Ia and the D4-ddUTP-extended primer (BTH274-15A). At a primer-to-template ratio ranging from 1000 to 16000, the normalized ratio in general remained at constant values of 1.73 and 4.0 for BTH274-15a and BAC303-5a, respectively. For BTH584-16a, the normalized ratio slightly decreased from 3.49 to 2.69. Based on these observations, the normalized ratios can be used further to calculate the concentration of those extended primers inside a HOPE reaction when the initial concentrations of oligonucleotide primers are present in excess of the template concentration (>1000 fold) to ensure a consistent primer extension efficiency.

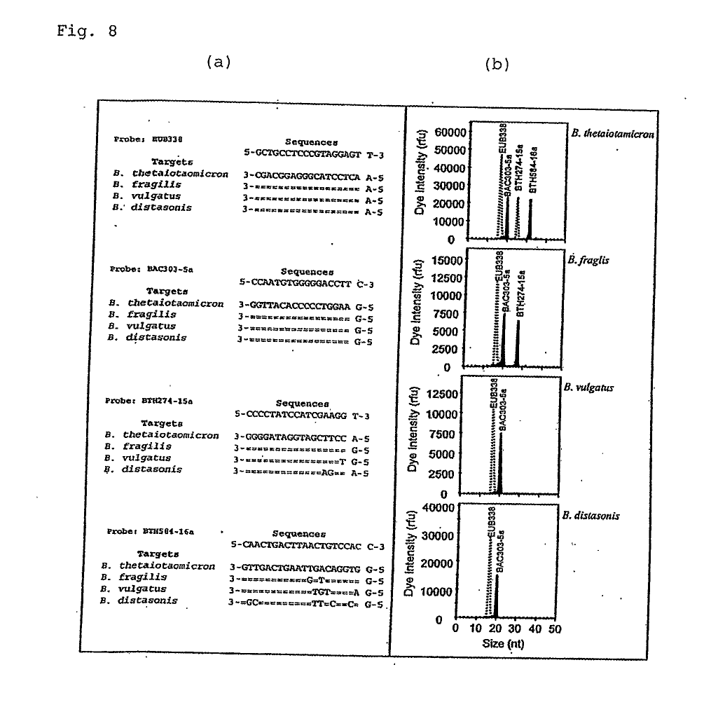

FIG. 8 shows the specificity of the HOPE method. The specificity of HOPE was first validated using four different Bacteroides species (i.e. B. thetaiotaomicron, B. fragilis, B. vulgatus and B. distasonis), and four different primers, EUB338, BAC303-5A, BTH584-16A and BTH274-15A, in a single reaction. FIG. 8a indicates that all four Bacteroides species were correctly extended with a D4-ddUTP or a D2-ddCTP by using EUB338 or BAC303-5A, respectively. Using BTH274-15A, B. thetaiotaomicron and B. fragilis were extended with a D4-ddUTP and a D2-ddCTP, respectively. Although exhibiting similar electrophoretic sizes, the D4-ddUTP- and D2-ddCTP-extended primers could be easily discriminated by the DNA sequencer based on the color of the dye. B. vulgatus and B. distasonis gave no fluorescence signals for primer BTH274-15A, as they contained a mismatch nucleotide “T” and two mismatch nucleotides “AG” near the 3′ end of the primer, respectively. Only B. thetaiotaomicron was successfully extended with a D2-ddCTP using BTH584-16A. No signal was observed with the other three Bacteroides species, as they contained at least two nucleotide mismatches to the sequence of BTH584-16A. These observations matched the in silico prediction as shown in FIG. 8b. Overall results suggested high discriminative ability of the HOPE technique with mismatched targets.

The specificity of the 4-plexing HOPE was further confirmed using 16 other reference species commonly found in the fecal samples (Table 1a). The HOPE reaction exhibited 100% accuracy in extending the correct type of nucleotides from those reference strains and the results matched the in silico prediction.

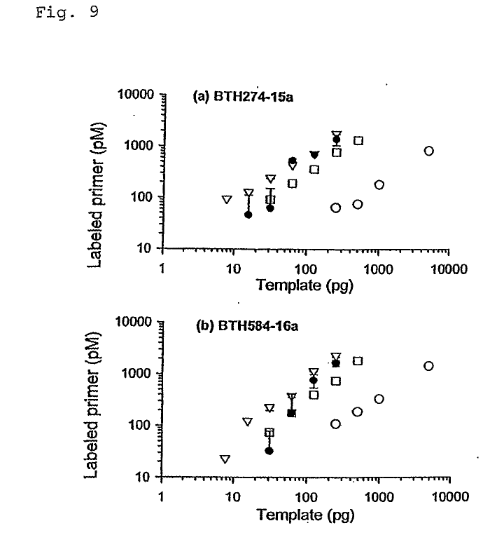

FIG. 9 shows the sensitivity of the HOPE method. The

HOPE reaction was carried out with different amounts (1-5 ng) of DNA template (i.e. B. thetaiotaomicron 165 rRNA gene) under three different reaction volumes (i.e., 5 μL, 10 μL and 20 μL). Two specific primers; BTH274-15A and BTH584-16A, were used. FIG. 9(a) indicates that the amount of the D4-ddUTP-extended BTH274-15A decreased linearly along with a decrease in the template concentration. Using a reaction volume of 20 μL, 10 μL and 5 μL, the lowest detectable amount of template was found to be 250 pg, 31.3 pg and 7.8 pg, respectively (R²=0.999, 0.993, and 0.988, respectively). These detectable amounts corresponded to 227 amol, 28.4 amol, and 7.1 amol, respectively. Similar results were obtained for primer BTH584-16a (FIG. 9b). The lowest amounts of templates detected were also 250 pg, 31.3 pg, and 7.8 pg for a reaction volume of 20 μL, 10 μL and 5 μL, respectively (R²=0.999, 0.991, and 0.994, respectively). It was clear that a small reaction volume at 5 μL could enhance detection sensitivity and reduce reagent usage.

The detection sensitivity of HOPE was further investigated by mixing a target template (i.e. B. thetaiotaomicron) with other targets present at high concentrations. In a 5-μL reaction volume, 100 ng of L. acidophilus 16S rRNA gene amplicons was mixed with different amounts (1-250 pg) of B. thetaiotaomicron 16S rRNA gene. The lowest detected amount was 15.6 pg for BTH274-15a or 31.3 pg for BTH584-16a (FIG. 9), and was comparable to previous data obtained without the presence of L. acidophilus amplicons in the DNA template. The minimum detectable target DNA was approximately 0.016-0.031% of the total DNA template at an amount of 100 ng.

Five different model communities were prepared individually and determined by the HOPE reaction as shown in Table 2. They comprise various amounts of 16S rRNA genes amplified from three different Bacteroides species, L. acidophilus, E. faecium, and P. productus, E. coli and M. barkeri. B. thetaiotaomicron could be simultaneously detected by all four primers (BTH274-15A, BTH584-16A, BAC303-5A, and EUB338Ia) in a HOPE reaction. B. distasonis and B. vulgatus could only be detected by BAC303-5a and EUB338Ia. The remaining four bacterial species could be detected by EUB338Ia, and M. bakeri could not be detected by any primers. The abundance of a specific target was calculated according to Equation (2). In MC1, MC2 and MC3, the observed ratios of Bacteroides by BAC303-5A were 77.0±3.9%, 49.2±3.6%, and 14.8±1.0% of total amplified DNA (molar basis), respectively, and were very close to the prepared ratios, 72.7%, 46.2% and 21.5%, respectively (Table 2). The ratio of B. thetaiotaomicron detected by BTH274-15A and BTH584-16A, which were 17.6-18.5% in MC1 and MC2, respectively, were also close to the prepared ratio (21.5-22.1%). However, a lower ratio (14.4-14.9%) was observed in MC3. In MC4, the abundance of Bacteroides species was observed to be 9.7% of total bacterial 16S rRNA gene, and was also very close to the theoretical value of 11.1%. The abundance of B. thetaiotaomicron detected by the primer BTH2840-15A and BTH584-16A was 2.8-3.4% of total bacterial 16S rRNA gene, and was close to the theoretical ratios. In MC5, by replacing E. coli 16S rRNA gene with M. barkeri 16S rRNA gene, the observed ratio of all Bacteroides species increased from 9.7% to 53.1% of total bacterial 16S rRNA gene. Accordingly, the abundance of B. thetaiotaomicron also increased from 2.8-3.4% to 15.3-17.4% of total bacterial 16S rRNA gene. These results showed that the HOPE reaction was able to accurately and reproducibly profile the relative abundance of selected targets in a pooled 16S rRNA gene sample.

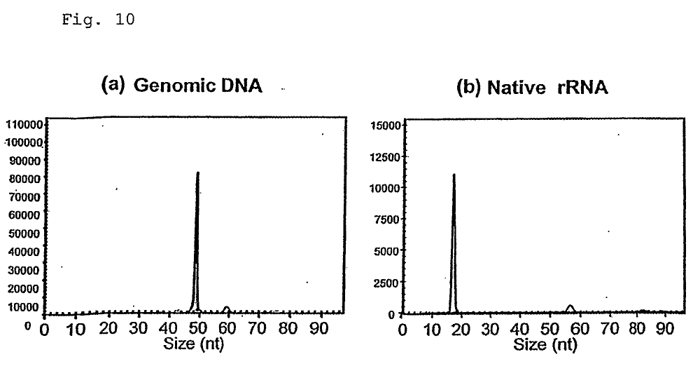

The types of target nucleic acids that can be analyzed in the HOPE reaction is shown in FIG. 10. In addition to the PCR amplified products, genomic DNA and native RNA can be used for analysis. For genomic DNAs as targets, longer primers (e.g. 25˜50 nt) were used to increase the primer extension efficiency, as shown in FIG. 10a. For native RNA targets, RNA-dependent DNA polymerase or reverse transcriptase was used, as shown in FIG. 10b. Analysis of RNA targets using the HOPE reaction was shown to be able to provide direct quantification of absolute copies of the targets in the samples.

To achieve multiplexing capability >4-plexing, a 6-plexing reaction and a 7-plexing reaction were designed and tested. FIG. 4 shows the primer combination in a 6-plexing HOPE reaction containing a domain Bacteria-specific primer and four group-specific primers (BTT1250, BAC303-5a, BTH274-15a, EUB338Ia-23a and BFRG602-19a). Depending on the targets, BTH274-15a can be extended with a ddCTP or a ddTTP. The 7-plexing reaction included two group-specific primers (BFRG602-19a and BTH274-15a), and four species-specific primers (BUFM1018-18a, BADF1037-9a, BFG1024 and BITT141).

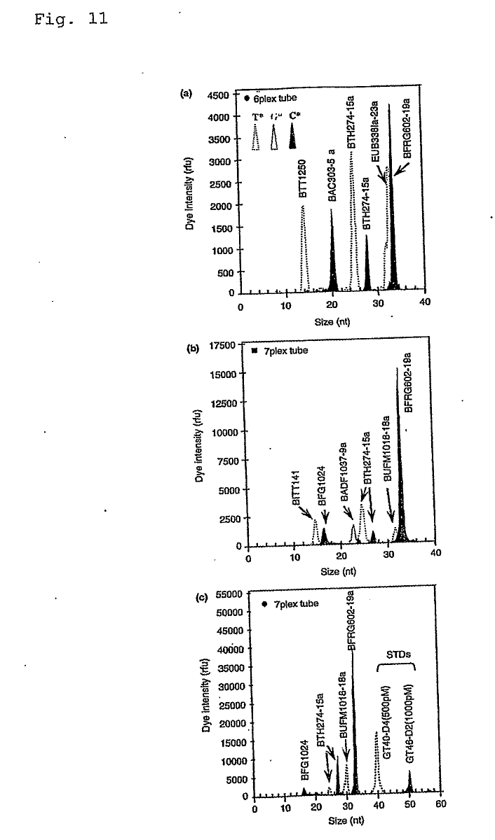

FIGS. 11a and 11b show the electrophoregrams of the 6-plexing and 7-plexing reactions, respectively, with different reference strains. Individual primers are clearly separated and identified based on the lengths and extended dye-terminator types. The calibration factor obtained between a given primer and a reference primer is comparable to that obtained from FIGS. 6 and 7.

The 6-plexing and 7-plexing HOPE reactions were further used together (in total, 10-plexing) to simultaneously determine the relative abundance of different Bacteroides spp. in the influent and effluent of a domestic wastewater treatment plant, where more than 25-34 detectable bacterial groups as determined by T-RFLP were observed (data not shown). FIG. 11c illustrates the electrophoregram obtained for the influent sample using the 7-plexing HOPE reaction. Those distinct detectable peaks correctly correspond to group-specific primers (BFRG602_{—}19a and BTH274-15a), two species-specific primers (BUFM1018-18a and BFG1024), and two size and concentration standards. Table 3 indicates that the relative abundance of the Bacteroidales group and BFRG602-related group accounts for 10-11.1% and 3.6-4.5%, respectively, of the amplified 16S rRNA genes in the influent samples. These percentages decreased to 1.1-1.6% and 50.1% in the effluent samples, a 7-45-fold reduction in the relative abundance. The BTH274-related group represented approximately 0.3-1.1,% of total amplified bacterial 16S rRNA genes in the influent samples, and was not detectable in the effluent. Within the BTH274-related group, B. fragilis and Bacteroides uniformis were present at 4.9-5.1% and 19.7-21.6% of the BFRG602-related group, or 0.2% and 0.8-0.9% of EUB338Ia detected bacteria, respectively, suggesting the presence of other Bacteroides spp. in the influent that were not targeted by the species-specific primers used.

### Example 2

### Quantitative Analyses of Human-Specific Bacteroides spp. in Feces and Wastewaters Using Hierarchical Oligonucleotide Primer Extension (HOPE) Reactions

This example demonstrates the use of HOPE as a new approach to perform quantitative analyses on 17 different human-specific Bacteroides spp. present in feces and wastewaters. By arranging 23 primers that are of different lengths and/or extend with different ddNTPs into seven HOPE reaction tubes, a high-throughput and rapid analysis was achieved within 90 min.

### (a) Materials and Methods

### Bacterial Strains and Environmental Samples

19 reference bacterial strains obtained from Japan Collection of Microorganisms (Wako, Japan) or Bioresources Collection and Research Centre (Hsinchu, Taiwan) were used. They included B. distasonis (JCM5825), B. uniformis (JCM5828), B. pyogenes (JCM6294), B. helcogenes (JCM6297), B. stercoris (JCM9496), B. merdae (JCM9497), B. caccae (JCM9498), B. tectus (JCM10003), B. acidifaciens (JCM10556), B. fragilis (BCRC10619), B. thetaiotaomicron (BCRC10624), B. vulgatus (BCRC12903), B. coprocola (JCM12979), B. massiliensis (JCM12892), B. eggerthii (JCM12986), B. nordii (JCM12987), B. salyersiae (JCM12988), B. intestinalis (JCM13266), and B. goldsteinii (JCM13446). Fecal samples were collected from (i) three healthy donors of age 26-32 years old, (ii) three healthy swines, and (iii) three healthy bovines. Environmental samples were collected from the influent of a municipal treatment plant located in Singapore and a swine wastewater treatment plant in Tainan, Taiwan. Samples were collected and kept at −20° C. prior to DNA extraction.

### DNA Extraction

Total DNA of pure cultures and influent samples was extracted according to a previously described protocol with minor modifications (Schmidt, T. M., E. F. DeLong, and N. R. Pace. 1991. Analysis of a marine picoplankton community by 16S rRNA gene cloning and sequencing. J Bacteriol 173:4371-8). Total DNA of fecal samples was extracted using QIAamp DNA stool mini kit (Qiagen), as this commercial kit was reported to be most suitable for DNA extraction from fecal samples.

PCR Amplification of 16S rRNA Genes

Each PCR reaction contained 50 to 100 ng of genomic DNA in 1× Takara Ex-Taq buffer, 200 nM of forward primer [11F, 5′-GTT TGA TCC TGG CTC AG-3′] and reverse primer [1492R, GGY TAC CTT GTT ACG ACT T-3′], 200 mM of dNTP and 0.5 U of Ex-Taq DNA polymerase (Takara).

The optimal number of thermal cycles required to obtain a proportional amplification of the microbial community was examined by varying the cycle number of thermal program (denaturation, 95° C. for 30 s; annealing, 55° C. for 45 s; and extension, 72° C. for 60 s) from 10 to 35 cycles at an increment of 5 cycles. Amplicons obtained at each cycle were concentrated and purified using QIAquick PCR purification kit (Qiagen), and the concentrations quantified by UV absorbance measurement using DU730 spectrophotometer (Beckman Coulter).

### Cloning and Sequencing

To ensure culture purity, 16S rRNA genes of those reference strains were separately cloned into the pCRII vector using the TA cloning kit (Invitrogen Corporation, Carlsbad, Calif., USA). For each clone library, 20 colonies were selected, and the presence of DNA insertion was confirmed by direct PCR amplification with M13R and M13F primers. The inserted DNA fragment was then sequenced using ABI PRISM 3.130 genetic analyzer (Applied Biosystems) and Bigdye Sequencing kit (Applied Biosystems). These sequences were compared against the 16S rRNA gene database using BLAST software.

### Species-Specific Primer Design

A total of 17 primers specifically targeting different human-specific Bacteroides spp. were designed using the probe design function of ARB software. An updated ssu_jan04.arb database (www.arb-home.de), which contained 28,289 nearly complete 16S rRNA sequences (length>1450 nt), was used together with 189 aligned sequences of cultivated Bacteroides spp. obtained from the Ribosomal Database Project II (RDP) and nearly complete 16S rRNA sequences of the 19 reference bacterial strains. To enhance the specificity of HOPE primers, each primer was designed with mismatch positions, of non-targets located at the 3′-terminus. The specificity of designed primers was first verified in silico against RDP database, and subsequently in HOPE reactions to ensure that primers do not extend when non-target bacterial strains were used as DNA template.

### Internal Standard Oligonucleotide Mixture

The mixture contained four oligonucleotides of different lengths [5′-(GT) x-3′; where x=18, 20, 22 and 24], which were synthesized and end-labeled at the 5′ end with four different fluorophores (i.e. dR110, dR6G, dTAMRA and dROX) (Applied Biosystems). The concentration of individual oligonucleotides was measured at 260 nm, and subsequently diluted to 3 nM for 5′-dR110-(GT) 20-3′, 15 nM for 5′-dTAMRA-(GT) 22-3′, 18 nM for 5′-dROX-(GT) 24-3′, and 3 nM for 5′-dR6G-(GT)18-3′.

### HOPE Reactions

Unless stated otherwise, each HOPE reaction (in total, 5 μl) contained 2.5 μl of SNaPshot premix, 5-30 fmol of DNA template, 10 pmole of oligonucleotide primers (Sigma-Proligo, Singapore and Mission Biotech, Taiwan), and various amounts of deionized water. The SNaPshot premix consisted of DNA polymerase, ionic buffer and fluorescently labeled dideoxynucleotides (dROXTM-ddTTP, dTAMRATM-ddCTP, dR110-ddGTP and dR6G-ddATP). HOPE thermal program consisted of 20 cycles of denaturation (96° C., 10 s), annealing (64° C., 30 s) and extension (72° C., 15 s). After primer extension reaction, 1 U of shrimp alkaline phosphatase (Roche Applied Science, Penzberg, Germany) was added for removal of unincorporated dye-terminators and incubated at 37° C. for 60 min. The enzyme was then inactivated at 75° C. for 10 min.

### Capillary Electrophoresis

0.5-1 μl of HOPE products were mixed with 0.125 μl of GeneScan Liz 120 standard (Applied Biosystems), 0.25 μl of the standard oligonucleotide mixture, and 12 μl of Hi-Di formamide (Applied Biosystems). The electrophoresis program included a denaturation step (60° C.), an injection step with an applied voltage of 1.0-2.1 kV for 12-40 s and a separation step. Fluorescence data were subsequently analyzed by the fragment analysis software GeneMapper, where the fragment sizes and peak areas of the extended primers and internal standard oligonucleotides were recorded.

### Calculation of Relative Abundance of HOPE Products

The concentration of extended primers was quantified as follows:

$$\frac{Peak area of extended primer}{( {C}_{p} ) ( volume of injected sample )} = \frac{ \begin{matrix}Peak area of associated \\ std oligonucleotide\end{matrix} }{( {C}_{std} ) ( volume of std )} \qquad ( i )$$

where Cₚ represents the molar concentration of a specific primer extended in the HOPE reactions for the samples; C_{std} represents the molar concentration of the associated standard oligonucleotide.

Calibration factors for each specific primer with respect to a higher ranked primer can be obtained using the associated reference strains as template, and calculated as follows:

$$C {F}_{A - B} = \frac{Concentration of extended primer A , {C}_{a}}{Concentration of extended primer B , {C}_{b}} \qquad ( ii )$$

where primer B is targeting at a higher hierarchical level compared to primer A.

The relative abundance of 16S rRNA gene amplicons targeted by primer A with respect to those targeted by primer B can then be calculated as follows:

$$Relative abundance of target ( \% ) = \frac{{C}_{a}}{{C}_{b} \times C {F}_{A - B}} \times 100 \% \qquad ( iii )$$

### (b) Results

### PCR Amplification

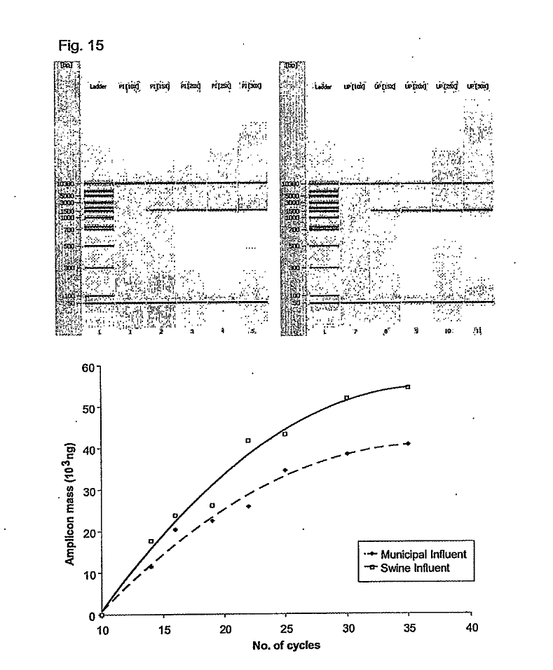

Proportional amplification of microbial targets was achieved when a 15-20-cycle PCR was carried out. Exponential amplification occurred after 20 cycles and reached a plateau after 35 cycles (FIG. 15). Therefore, for all PCR amplifications of microbial targets in fecal and wastewater samples, a 20-cycle PCR was chosen to generate DNA templates for subsequent HOPE analyses. This can minimize the bias introduced by PCR when performing quantitative analyses of species richness.

### Design of Hierarchical Primers

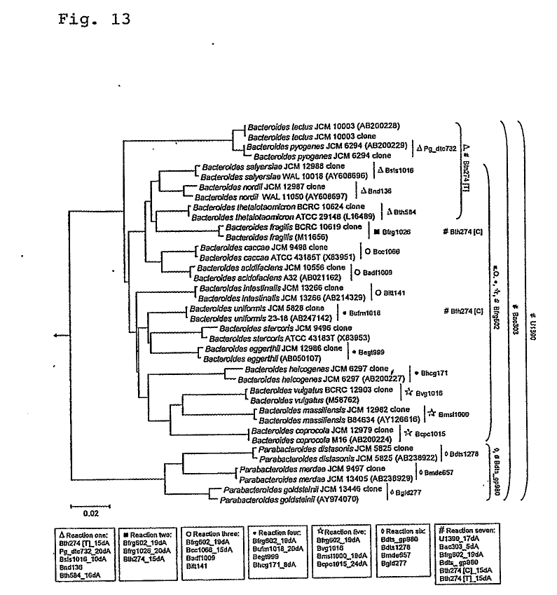

Table 4 lists the sequences and specificities of 23 different primers related to Bacteroides at species, group, or domain levels. Primers are assigned into seven different multiplexing HOPE reactions based on the phylogenetic affiliation of those bacterial strains (FIG. 13). Reactions one to six contained three higher ranking primers (Bth274, Bdts_gp980 or Bfrg602) together with 17 species-specific primers. Thus, the abundance of individual microbial targets targeted by each species-specific primer relative to those targeted by higher ranking primers can be quantified. In reaction seven, the relative abundance of microbial targets represented by Bth274, Bdts_gp980, Bfrg602 and Bac303 with respect to the total Bacteria (U1390-related) can be calculated. To clearly differentiate extended primers in the same HOPE reaction, primers that extend with the same ddNTP were modified with different lengths of poly-A tails from five to 24 at the 5′ end (Table 4).

### Specificity of Primer Extension

The specificities of primer extension were validated against reference strains. Results showed that all species-specific primers correctly extended with the same nucleotide as in silico predicted, when their targeted species were used as DNA template in the reaction.

Likewise, all species-specific primers did not extend when non-targets were present. Group-specific primer Bth274 extended with nucleotide “C” in the presence of B. uniformis and B. fragilis, or “T” in the presence of B. tectus, B. pyogenes, B. nordii, B. salyersiae and B. thetaiotaomicron. Bfrg602 was designed to target Bacteroides spp. in B. fragilis cluster, and extended with “C”. Bac303 and U1390, which were designed to target all 19 reference bacterial strains at the order and domain level respectively, extended with “C” and “A” respectively (Table 4).

### Interpretation of Electrophoregrams of HOPE Reactions

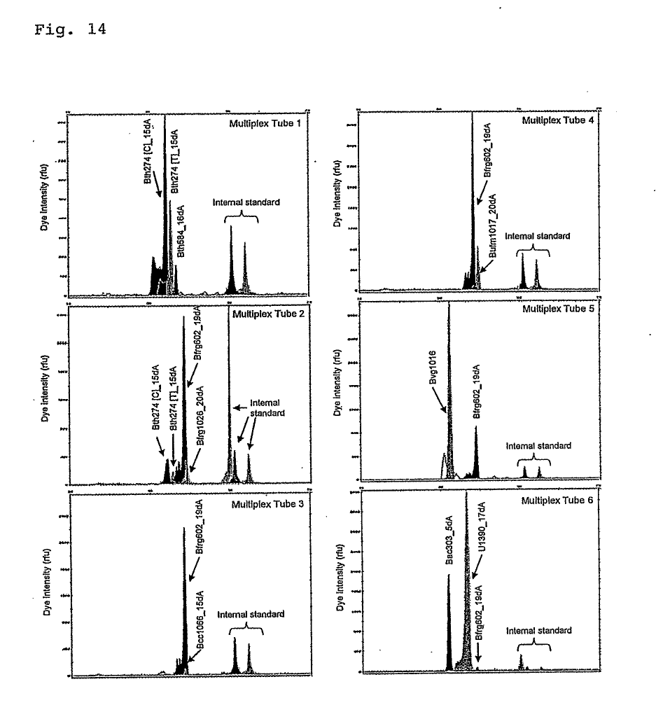

FIG. 14 shows the electrophoregrams for the extended primers representing different human-specific Bacteroides spp. in human feces. As higher-ranking primers have more targets to prime to, their peak height and area were considerably larger than those representing species-specific primers. Therefore in preferred embodiments of HOPE reactions with species-specific primers, the associated higher-ranking primer may not target beyond the family level. This is to facilitate the detection of extended species-specific primers with low peak heights. In addition, as extended primers would appear with a consistent fragment size, all other peaks that do not coincide with the extended primers may be treated as background noise and removed from subsequent analyses.

Peak areas of extended primers and internal standard oligonucleotides were noted and used for calculating relative abundance.

### Relative Abundance of Human-Specific Bacteroides spp. in Fecal Samples

Table 5 describes the relative abundances of human-specific Bacteroides spp. at various taxonomical levels. In all human fecal samples, B. vulgatus was observed as the dominant species (14.5 to 51.2%) among the Bfrg602-related group, but inter-individual differences in the diversity of Bacteroides were apparent. B. fragilis, B. eggerthii, B. intestinalis and B. massiliensis were detected to be between 0.6 and 6.8% in volunteers H2 and H3, but were too low to be detected in volunteer H1. B. caccae and B. uniformis were also present at varying relative abundances (1.3-9.1%). At the higher taxonomical levels, Bth274 [C]-related group (including B. uniformis and B. fragilis) accounted for 0.4 to 0.8% of Bacteria present in the fecal flora. Likewise, Bth274 [T]-related group (including B. thetaiotaomicron, B. tectus, B. caccae, B. pyogenes, B. nordii and B. salyersiae) accounted for 0.1 to 0.6% of Bacteria, with B. thetaiotaomicron as the predominant species (44.8 to 96.4%) in this group. Bfrg602-related cluster (B. fragilis cluster) varied across the host, accounting from 3.0 to 6.6% of Bacteria. It is further observed that the relative abundance of the Bacteroidales group targeted by Bac303 primer ranged from 14.3 to 21.8% among the three human volunteers, suggesting that human fecal flora has a relatively stable profile of dominant microorganisms. Species richness however varied distinctly from one individual to another, presumably due to differences in living habits, age and diet. Comparatively, the swine and bovine fecal flora had distinctly lower proportions of human-specific Bacteroides spp. No fragments representing extended Bth274_{—}15dA and Bfrg602_{—}19dA were detected in HOPE reaction seven. Bacteroidales targeted by Bac303_{—}5dA ranged from 2.4 to 5.1% of total Bacteria in the swine fecal flora and 1.8 to 2.9% of total Bacteria in the bovine feces. This relative abundance of Bacteroidales is more than five-fold lower than those in human fecal flora.

### Relative Abundance of Human-Specific Bacteroides spp. in Wastewaters

HOPE was further applied to examine the relative abundances of Bacteroides spp. in the influent of a swine and municipal wastewater treatment plant (Table 5). Bacteroides spp. that were previously detected in the human feces were also present in municipal wastewater, with B. fragilis, B. caccae, B. uniformis, B. vulgates and B. massiliensis accounting for 11.8%, 3.1%, 21.5%, 32.5%, and 6.1% of Bfrg602-related group respectively. B. thetaiotaomicron remained the predominant species in Bth274 [T]-related group, constituting about 93.6%. Although the relative abundance of Bth274-related and Bfrg602-related group that was present in the municipal wastewater was close to those found in human fecal flora, the relative abundance of amplified 16S rRNA genes in the order Bacteroidales appeared to have decreased by more than two-fold. This may suggest the low persistence of Bacteroides spp. in the environment. In the influent of swine wastewater treatment plant, none of the primers targeting Bacteroides spp. at the species-level could be detected. Relative abundance of targets in the order Bacteroidales was about three-fold lower than in municipal wastewater. This reaffirmed the previous observation that human-specific Bacteroides spp. is present in lower abundance in animal wastes.

### (c) Discussion

To study the functional importance of Bacteroides spp. in gut, molecular-based methods like FISH and the construction of 16S rRNA clone libraries have been used despite their limitations in performing quantification. Based on 16S rRNA clone libraries, phylum Bacteroidetes has been found to account for 27.7 to 48% of total Bacteria. Among them, B. fragilis and B. distasonis subgroups accounted for up to 13.3% of total Bacteria, indicating their dominance and importance in the well-functioning of human gut.

At the species level, B. thetaiotaomicron, B. vulgatus, B. uniformis and B. caccae have been determined by Suau et al. (Direct analysis of genes encoding 16S rRNA from complex communities reveals many novel molecular species within the human gut. Appl Environ Microbiol 65:4799-807v) to make up 2.1%, 1.4%, 4.9% and 1.1% of Bacteria, respectively. This reported abundance is however more than three-fold lower than those reported by Eckburg and coworkers (2005; Diversity of the human intestinal microbial flora. Science 308:1635-8), who found that B. vulgatus and B. thetaiotaomicron made up 15% and 6.2% of Bacteria, respectively (Table 6).

Differences in the reported abundances of Bacteroides spp. in these two studies may be a result of variations in amplification cycles prior to cloning, as well as the total number of clones picked for analysis. Likewise, FISH has shown similar abundance of Bacteroides at the subgroup and order level. However, no detailed studies to quantify each species in the genus Bacteroides were performed (Table 6). This may be due to inactivity of Bacteroides 16S rRNA genes in feces, resulting in low detection sensitivity.

Compared to the above-mentioned methods, HOPE has a detection sensitivity of 0.05-0.1% of the total PCR-amplified microbial target and is therefore capable of detecting microbial targets at the species-level. Using HOPE, the relative abundance of Bacteroidales was found to account for 14.3 to 21.8% of total Bacteria, and was similar to those reported by 16S rRNA clone libraries. However, the abundance of each Bacteroides spp. is more than three-fold lower (Table 6). Such variations in the relative abundance are likely to be due to discrepancies in the two methodologies. Unlike HOPE, which examines the entire PCR-amplified microbial community, quantitative analyses by means of clone libraries are dependent on the ultimate number of clones picked. Therefore, HOPE analyses may represent the actual species richness more accurately than clone libraries. In addition, the entire HOPE procedure can be completed in less than 90 min, thus providing a rapid and sensitive method for quantitative analyses in any sample type.

As HOPE can rapidly quantify Bacteroides spp. in both feces and contaminated waters, it can be used in different environmental microbiological studies. Since Kreader et al. (1995; Design and evaluation of Bacteroides DNA probes for the specific detection of human fecal pollution. Appl Environ Microbiol 61:1171-9) found that the abundance of human-specific Bacteroides spp. can distinguish human feces from non-humans', HOPE can be used to quantify these microbial targets and thus serve as a methodology for fecal source tracking (FST). This study showed the application of HOPE to quantify the relative abundance of Bacteroides spp. in samples that have a high proportion of them, and may not accurately reflect its ability to perform FST in large water bodies where the amount of Bacteroides 16S rRNA gene might be less than 0.1% with respect to total Bacteria. In such cases, to increase the detection sensitivity of HOPE, Bacteroides-Prevotella instead of Bacteria can be amplified out to serve as DNA template for HOPE analyses. By complementing this alternative HOPE-based FST strategy to the conventional fecal indicator tests, a better understanding of fecal contamination can be achieved.

Besides using HOPE for FST, the method can also be applied to identify disease-related biomarkers that are present in gut or feces. This can be achieved by first comparing the relative abundance of these biomarkers in healthy and diseased subjects. Coupled with the understanding of the functional roles played by these biomarkers, an alternative treatment strategy that involves the manipulation of the microbial diversity can thus be formulated. Till today, only culture-based and conventional molecular methods like denaturing gradient gel electrophoresis (DGGE) are applied to this field. HOPE has a comparative edge to these conventional methods, as it provides quantitative analyses and thus establishes a better statistical correlation between the identified biomarkers and human diseases.

In summary, this study has demonstrated the use of HOPE as a rapid and high-throughput detection method that is able to quantify microbial targets at various taxonomical levels. The versatility of this approach would also mean that it can be expanded to any microbial targets in a PCR-amplified community once appropriate hierarchical primer designs are available, thus facilitating further understanding of microbial diversity in different ecologies.

### APPLICATIONS

Advantageously, the disclosed method enables rapid, high throughput, sensitive and accurate detection, identification and/or quantification of a plurality of target nucleic acids in a sample in a single analysis. The present method has potential, applications in the development of commercialized or customized. diagnostic kits in environmental and clinical usages, including monitoring of indicator microorganisms in drinking water (e.g. pathogens, fecal bacteria and toxin-producing microorganisms), in the water and wastewater biological treatment processes (biofilm, filamentous bacteria and foam-forming bacteria), in the bioremediation of contaminated soil and ground water (indicator bacteria), and in the guts of human and animals (bacteria relevant to health and disease) where the abundances of specific populations or detection and/or quantification of multiple targets are important or required.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

TABLE 1a
Hierarchical oligonucleotide primer extension
analyses for 20 bacterial strains.

| | | | Eub338 (18 nt) | | | BAC303-5a (22 nt) | | | BTH274-15a (32 nt) | | | BTH584-16a (36 nt) | | |
|---|---|---|---|---|---|---|---|---|---|---|---|---|---|---|
| # | Species | Source^{a} | MM^{b} | Allele^{c} | ddNTP^{d} | MM | Allele | ddNTP | MM | Allele | ddNTP | MM | Allele | ddNTP |
| 1 | Bacteroides | BCRC10624 | 0 | A | U | 0 | G | C | 0 | A | U | 0 | G | C |
| | thetaiotaomicron | | | | | | | | | | | | | |
| 2 | Bacteroides fragilis | BCRC10619 | 0 | A | U | 0 | G | C | 0 | G | C | 2 | — | ND |
| 3 | Bacteroides distasonis | JCM5825 | 0 | A | U | 0 | G | C | 2 | — | ND | 6 | — | ND |
| 4 | Bacteroides vulgatus | BCRC12903 | 0 | A | U | 0 | G | C | 7 | — | ND | 4 | — | ND |
| 5 | Ruminococcus albus | DSMZ20455 | 0 | G | C | 6 | — | ND | 7 | — | ND | 8 | — | ND |
| 6 | Collinsella aerofaciens | JCM10188 | 0 | G | C | 6 | — | ND | 7 | — | ND | 10 | — | ND |
| 7 | Lactobacilllus | DSMZ20079 | 0 | A | U | 5 | — | ND | 7 | — | ND | 7 | — | ND |
| | acidophilus | | | | | | | | | | | | | |
| 8 | Bifidobacterium | BCRC14606 | 0 | G | C | 6 | — | ND | 5 | — | ND | 9 | — | ND |
| | adolescentis | | | | | | | | | | | | | |
| 9 | Peptostreptococcus | DSM2950 | 0 | G | C | 6 | — | ND | 8 | — | ND | 9 | — | ND |
| | productus | | | | | | | | | | | | | |
| 10 | Clostridium leptum | BCRC14522 | 0 | G | C | 6 | — | ND | 7 | — | ND | 10 | — | ND |
| 11 | Ruminococcus bromii | ATCC27255 | 0 | G | C | 6 | — | ND | 7 | — | ND | 8 | — | ND |
| 12 | Bifidobacterium | BCRC11847 | 0 | G | C | 6 | — | ND | 5 | — | ND | 9 | — | ND |
| | longum | | | | | | | | | | | | | |
| 13 | Enterococcus faecium | BCRC10067 | 0 | A | U | 5 | — | ND | 6 | — | ND | 7 | — | ND |
| 14 | Clostridium | BCRC14545 | 0 | A | U | 6 | — | ND | 6 | — | ND | 8 | — | ND |
| | clostridiiforme | | | | | | | | | | | | | |
| 15 | Bifidobacterium | DSMZ20088 | 0 | G | C | 6 | — | ND | 5 | — | ND | 9 | — | ND |
| | longum | | | | | | | | | | | | | |
| 16 | Ruminococcus abeum | ATCC29174 | 0 | G | C | 6 | — | ND | 7 | — | ND | 6 | — | ND |
| 17 | Eubacterium biforme | DSMZ3989 | 0 | A | U | 6 | — | ND | 5 | — | ND | 9 | — | ND |
| 18 | Fusobacterium | ATCC27768 | 0 | G | C | 5 | — | ND | 6 | — | ND | 6 | — | ND |
| | prausnitzii | | | | | | | | | | | | | |
| 19 | Ruminococcus callidus | ATCC27760 | 0 | G | C | 5 | — | ND | 8 | — | ND | 7 | — | ND |
| 20 | Escherichia coli | NCIMB10083 | 0 | G | C | 6 | — | ND | 8 | — | ND | 5 | — | ND |

^{a}Bacterial strains are obtained from American Type Culture Collection (ATCC), Bioresource Collection and Research Center (BCRC), Japan Collection of Microorganisms (JCM), German National Resource Centre for Biological Material (DSMZ) and National Collection of Industrial, Marine and Food Bacteria (NCIMB).
^{b}Numbers of the mismatched base-pairing (MM).
^{c}Refer to the nucleotides of the template adjacent to the primer-template duplex formed.
^{d}Refer to the dye-terminators observed.
ND, not detected.

TABLE 1b
Sequences and specificity of the oligonucleo-
tides used in the study

| | | |
|---|---|---|
| Name | Sequence (5′-3′) | Specificity |

Oligonucleotide primers

| | | |
|---|---|---|
| EUB338 | GCTGCCTCCCGTAGGAGT | Bacteria |
| EU8338-4a | (A)₄ GCTGCCTCCCGTAGGAGT | Bacteria |
| EUB338-8a | (A)₈ GCTGCCTCCCGTAGGAGT | Bacteria |
| EUB338-12a | (A)₁₂ GCTGCCTCCCGTAGGAGT | Bacteria |
| EU8338-18a | (A)₁₈ GCTGCCTCCCGTAGGAGT | Bacteria |
| EUB338-24a | (A)₂₄ GCTGCCTCCCGTAGGAGT | Bacteria |
| EUB338-30a | (A)₃₀ GCTGCCTCCCGTAGGAGT | Bacteria |
| EUB338Ia | GCTGCCTCCCGTAGGAG | Bacteria |
| EUB338Ia- | (A)₂₃ GCTGCCTCCCGTAGGAG | Bacteria |
| 23a | | |
| BAC303-5a | (A)₅ CCAATGTGGGGGACCTT | Bacteroidales |
| | | group |
| BTH274-15a | (A)₁₅ CCCCTATCCATCGAAGG | B. |
| | | thetaiotao- |
| | | micron, |
| | | B. fragilis |
| | | and other |
| | | Bacteroides^{a} |
| BTH584-16a | (A)₁₆ CAACTGACTTAACTGTCCAC | B. |
| | | thetaiotao- |
| | | micron |
| BFRG602- | (A)₁₉ GAGCCGCAAACTTTCACAA | B. fragilis |
| 19a | | cluster^{a} |
| BTT1250 | CGCCGTGTAGCAACCTGC | Btt1250 |
| | | group^{a} |
| BFRG1024 | TCACAGCGGTGATTGCTCA | B. fragilis |
| BITT141 | CGAAAGGCTATCCCGGAA | Bacteroides |
| | | intestinalis |
| BADF1037- | (A)₉ TGCAGCACCTTCACACCT | Bacteroides |
| 9a | | acidifaciens |
| BUFM1018- | (A)₁₈ AACTGCCTTGCGGCTGACA | Bacteroides |
| 18a | | uniformis |

Fluorophore-labeled oligonucleotide standards

| | |
|---|---|
| GT16-Cy5 | Cy5-(GT)₈ |
| GT26-Cy5 | Cy5-(GT)₁₃ |
| GT36-Cy5 | Cy5-(GT)₁₈ |
| GT46-Cy5 | Cy5-(GT)₂₃ |
| GT46-D2 | D2-(GT)₂₃ |
| GT40-D4 | D4-(GT)₂₀ |

^{a}Refer to FIG. 4.

TABLE 2
Quantitative analyses of the model communities composed of defined 16S rRNA
gene amplicons using multiplexing HOPE method.

| | Concentration of 16S rRNA gene amplicons | | | | | | | |
|---|---|---|---|---|---|---|---|---|
| Model | prepared in the model communities¹ | | | | | | | |
| communities | B. thetaiotaomicron | B. distasonis | B. vulgatus | L. acidophilus | E. faecium | P. productus | E. coli | M. barkeri |
| MC1 | 2.2 | 2.5 | 2.5 | 2.7 | 0 | 0 | 0 | 0 |
| | (22.1%) | (24.8%) | (25.7%) | (27.3%) | | | | |
| MC2 | 2.2 | 2.5 | 0 | 2.7 | 2.7 | 0 | 0 | 0 |
| | (21.8%) | (24.4%) | | (26.8%) | (26.9%) | | | |
| MC3 | 2.2 | 0 | 0 | 2.7 | 2.7 | 2.7 | 0 | 0 |
| | (21.5%) | | | (26.5%) | (26.6%) | (25.3%) | | |
| MC4 | 1.1 | 1.2 | 0 | 1.3 | 1.4 | 0 | 15.8 | 0 |
| | (5.2%) | (5.9%) | | (6.5%) | (6.5%) | | (75.9%) | |
| MC5 | 1.1 | 1.2 | 0 | 1.3 | 1.4 | 0 | 0 | 12.8 |
| | (21.8%) | (24.4%) | | (26.8%) | (26.9%) | | | (0%) |

| | % of primers for specific groups with respect | | | | | |
|---|---|---|---|---|---|---|
| | to primer EUB3381a for | | | | | |
| | domain Bacteria (mean ± sd)² | | | | | |
| | BAC303-5a | | BTH274-15a | | BTH584-16a | |
| Model | (for Bacteroides) | | (for B. thetaiotaomicron) | | (for B. thetaiotaomicron) | |
| communities | Theoretical | Observed | Theoretical | Observed | Theoretical | Observed |
| MC1 | 72.7 | 77.0 ± 3.9 | 22.1 | 18.4 ± 0.7 | 22.1 | 18.5 ± 1.2 |
| MC2 | 46.2 | 49.2 ± 3.6 | 21.8 | 17.6 ± 0.7 | 21.8 | 18.1 ± 1.4 |
| MC3 | 21.5 | 14.8 ± 1.0 | 21.5 | 14.4 ± 0.9 | 21.5 | 14.9 ± 1.0 |
| MC4 | 11.1 | 9.7 ± 0.8 | 5.2 | 2.8 ± 0.3 | 5.2 | 3.4 ± 1.0 |
| MC5 | 46.2 | 53.1 ± 2.9 | 21.8 | 17.4 ± 0.4 | 21.8 | 15.3 ± 1.2 |

¹femto-mol/μl; percentages in the brackets indicated species abundance in the Bacteria domain.

TABLE 3
Quantifying relative abundance of specific
targets in the influent and effluent from sewage treatment
plant using multiplexing HOPE method

| Target abundance within a group | | Influent (%) | | Effluent (%) | |
|---|---|---|---|---|---|
| Target | Group | Sample 1 (n = 3) | Sample 2 (n = 3) | Sample 1 (n = 3) | Sample 2 (n = [symbol] |
| Bacteroidales | Bacteria (EUB3381a-23a) | 11.1 ± 1.4 | 10.0 ± 0.3 | 1.6 ± 0.1 | 1.1 ± 0.9 |
| BFRG602-related group | | 1.1 ± 0.9 | 3.6 ± 0.5 | ND | 0.1 ± 0.1 |
| BTH274c-related group^{a} | | 1.1 ± 0.1 | 0.8 ± 0.2 | ND | ND |
| BTH274t-related group^{b} | | 0.5 ± 0.1 | 0.3 ± 0.3 | ND | ND |
| BTT1250-related group | | ND | ND | ND | ND |
| B. fragilis | BFRG602-related group | 4.9 ± 0.7 | 5.1 ± 1.2 | ND | ND |
| B. uniformis | | 19.7 ± 1.0 | 21.6 ± 1.1 | ND | ND |
| B. intestinalis | | ND | ND | ND | ND |
| B. acidifaciens | | ND | ND | ND | ND |

^{a}The group detected by primer extension with ddCTP of BTH274-15a.
^{b}The group detected by primer extension with ddTTP of BTH274-15a.
Calibration factors obtained for the 6-plexing, EUB338Ia-23a: BAC303-5a: BFRG602-19a: BTH274-15a(C): BTH274-15a(T): BTT1250 = 1: 6.1: 11.2: 14.4: 3.1: 2.4, and 7-plexing, BFRG602-19a: BUFM1018-18a: BTH274-15a(C): BTH274-15a(T): BFG1024: BITT141 = 1: 0.8: 1.2: 0.4: 1.7: 0.1.
Bacterial strains used include B. thetaiotaomicron, B. fragilis, B. acidifaciens (JCM10556), B. intestinalis (JCM13266), B. uniformis (JCM5828), Bacteroides tectus (JCM10003), and Bacteroides pyogenes (JCM6294).
ND, not detected. [symbol]
indicates data missing or illegible when filed

TABLE 4

Primers included in this study are designed to
target human-specific Bacteroides spp. at various
hierarchical levels (species, genus, order and domain
level).

| | | | | | |
|---|---|---|---|---|---|
| | | | Poly-A | Type of | |
| | | | tail | ddNTP | |
| Primer | Target | Sequence (5′-3′) | (nt) | added | Reference |

| | | | | | |
|---|---|---|---|---|---|
| Pg_dtc 732 | B. pyogenes | GTT CCG GCC CGG TGA GCT | 20 | G | This study |
| Bhcg 171 | B. helcogenes | TTT CAG TGC CAT CGG GCA T | 8 | T | This study |
| Bcc 1066 | B. caccae | CGT ATG GGT TTC CCC ATA A | 15 | T | This study |
| Badf 1009 | B. acidifaciens | CGG CTA ACA TGT TTC CAC | 0 | A | This study |
| Bvg 1016 | B. vulgatus | ATG CCT TGC GGC TTA CGG C | 0 | T | This study |
| Bcpc 1015 | B. coprocola | CGC CTT GCG GCT TAC AAG T | 24 | T | This study |
| Bmsl 1000 | B. massiliensis | GCG TTT CCG CCA TAT TCG G | 19 | T | This study |
| Begt 999 | B. eggerthii | GTT TCC ACT ACA TTC CGC | 0 | T | This study |
| Bnd 136 | B. nordii | AGC CTA TCC CCG AGT AAA A | 0 | G | This study |
| Bsls 1016 | B. salyersiae | GCC TTG CGG CTA TGC CG | 10 | T | This study |
| Bmde657 | B. merdae | TCC GCC TAC CTC AAA CAC | 0 | A | This study |
| Bgld277 | B. goldsteinii | GAA CCC CTA TCC ATC GTG | 8 | G | This study |
| Bdts1278 | B. distasonis | AGA CGT GGT TTG GGG ATT | 0 | C | This study |
| Bufm 1018 | B. uniformis | CTG CCT TGC GGC TGA CA | 20 | T | (32) |
| Bfrg 1026 | B. fragilis | TCA CAG CGG TGA TTG CTC | 20 | A | (32) |
| Bth 584 | B. thetaiotaomicron | CAA CTG ACT TAA CTG TCC AC | 16 | C | (32) |
| Bitt 141 | B. intestinalis | CGA AAG GCT ATC CCG GAA | 0 | T | (32) |
| Bdts_gp980 | B. distasonis | CGT TCA AAC CCG GGT AA | 12 | G | This study |
| | sub-group | | | | |
| Bth 274 | B. fragilis | CCC CTA TCC ATC GAA GG | 15 | C/T | (32) |
| | sub-group | | | | |
| Bfrg 602 | B.fragilis group | GAG CCG CAA ACT TTC ACA A | 19 | C | (14) |
| Bac 303 | Bacteroidales | CCA ATG TGG GGG ACC TT | 5 | C | (26) |
| UI390 | Bacteria | YGA CGG GCG GTG TGT | 17 | A | (38) |

(14) Harmsen, et al. 2002. Extensive set of 16S rRNA-based probes for detection of bacteria in human feces. Appl Environ Microbiol 68: 2982-90.
(26) Manz, et al. 1996. Application of a suite of 16S rRNA-specific oligonucleotide probes designed to investigate bacteria of the phylum cytophaga-flavobacter-bacteroides in the natural environment. Microbiology 142 (Pt 5): 1097-106.
(32) Wang, et al. 1996. PCR detection and quantitation of predominant anaerobic bacteria in human and animal fecal samples. Appl Environ Microbiol 62: 1242-7.
(38) Zheng, et al. 1996. Characterization of universal small-subunit rRNA hybridization probes for quantitative molecular microbial ecology studies. Appl Environ Microbial 62: 4504-13.

TABLE 5
Relative abundance of microbial targets in the human, swine and cow fecal
samples together with those present in the influent of a municipal and swine WWTP were
quantified individually using multiplexing HOPE method.

| | | H1 | H2 | H3 | P1 | P2 | P3 |
|---|---|---|---|---|---|---|---|
| Target | Group | Relative abundance (%) | | | | | |
| B. fragilis | Bfrg602- | ND³ | 6.8 ± 0.8 | 6.5 | N.D. in | | |
| | related group | | | 0.5± | preliminary | | |
| B. caccae | | 9.1 ± 1.3 | 1.3 ± 0.7 | 2.0 ± 0.3 | testing | | |
| B. acidifaciens | | ND | ND | ND | | | |
| B. intestinalis | | ND | ND | 1.4 ± 0.2 | | | |
| B. uniformis | | 9.1 ± 1.1 | 4.0 ± 0.6 | 7.6 ± 0.5 | | | |
| B. eggerthii | | ND | 0.6 ± 0.1 | ND | | | |
| B. helcogenes | | ND | ND | ND | | | |
| B. vulgatus | | 27.9 ± 1.5 | 14.5 ± 1.0 | 51.2 ± 3.1 | | | |
| B. massiliensis | | ND | 2.9 ± 1.5 | ND | | | |
| B. coprocola | | ND | ND | ND | | | |
| B. nordii | Bth274 [T]- | ND | ND | ND | N.D. in | | |
| B. salyersiae | related | ND | ND | ND | preliminary | | |
| B. pyogenes | group | ND | ND | ND | testing | | |
| B. thetaiotaomicron | | 61.1 ± 0.3 | 44.8 ± 2.0 | 96.4 ± 4.1 | | | |
| Bacteroidales | U1390- | 14.3 ± 1.3 | 21.8 ± 1.7 | 14.7 ± 1.1 | 2.4 ± 0.2 | 4.6 ± 0.9 | 5.1 ± 0.7 |
| Bfrg602-related | related domain | 3.0 ± 0.2 | 6.3 ± 0.3 | 6.6 ± 0.2 | ND | ND | ND |
| group | | | | | | | |
| Bth274 [C]- | | 0.4 ± 0.1 | 0.7 ± 0.1 | 0.8 ± 0.2 | ND | ND | ND |
| related | | | | | | | |
| group¹ | | | | | | | |
| Bth274 [T]- | | 0.6 ± 0.1 | 0.3 ± 0.0 | 0.1 ± 0.0 | ND | ND | ND |
| related | | | | | | | |
| group² | | | | | | | |

| | | | | | Municipal | |
|---|---|---|---|---|---|---|
| | | | | | WWTP | Swine WWTP |
| | | C1 | C2 | C3 | Influent | Influent |
| Target | Group | Relative abundance (%) | | | | |
| B. fragilis | Bfrg602- | N.D. in | | | 11.4 ± 1.8 | N.D. in |
| | related group | preliminary | | | | preliminary |
| B. caccae | | testing | | | 3.1 ± 0.8 | testing |
| B. acidifaciens | | | | | ND | |
| B. intestinalis | | | | | ND | |
| B. uniformis | | | | | 21.5 ± 1.9 | |
| B. eggerthii | | | | | ND | |
| B. helcogenes | | | | | ND | |
| B. vulgatus | | | | | 32.5 ± 3.5 | |
| B. massiliensis | | | | | 6.1 ± 0.3 | |
| B. coprocola | | | | | ND | |
| B. nordii | Bth274 [T]- | N.D. in | | | ND | N.D. in |
| B. salyersiae | related | preliminary | | | ND | preliminary |
| B. pyogenes | group | testing | | | ND | testing |
| B. thetaiotaomicron | | | | | 93.6 ± 6.6 | |
| Bacteroidales | U1390- | 2.3 ± 0.3 | 2.9 ± 0.2 | 1.8 ± 0.2 | 5.5 ± 1.6 | 2.0 ± 0.2 |
| Bfrg602-related | related domin | ND | ND | ND | 3.7 ± 1.4 | ND |
| group | | | | | | |
| Bth274 [C]- | | ND | ND | ND | 0.7 ± 0.3 | ND |
| related | | | | | | |
| group¹ | | | | | | |
| Bth274 [T]- | | ND | ND | ND | 0.13 ± 0.07 | ND |
| related | | | | | | |
| group² | | | | | | |

¹Primer Bth274_15dA extends by ddCTP when annealed to microbial targets in this group.
²Primer Bth274_15dA extends by ddTTP when annealed to microbial targets in this group.
³ND denotes not detected.
Calibration factors (CF) obtained for primers in multiplexing tubes 1-4, Bfrg602_19dA: Bfrg1026_20dA: Bcc1066_15dA: Badf1009: Bitt141: Bufm1018_20dA: Begt999: Bhcg171_8dA: Bvg1016: Bms11000_19dA: Bcpc1015_24dA = 1: 0.17: 2.96: 0.36: 16.51: 4.61: 8.17: 13.86: 8.37: 3.13: 3.06. CF were obtained using clones from bacterial strains that includes B. fragilis (BCRC10619), B. caccae (JCM9498), B. acidifaciens (JCM10556), B. uniformis (JCM5828), B. eggerthii (JCM12986), B. helcogenes (JCM6297), B. vulgatus (BCRC12903), B. massiliensis (JCM12982) and B. coprocola (JCM12979).
Calibration factors (CF) obtained for primers in multiplexing tube 5, Bth274 [T]_15dA: Bnd136: Bsls1016_10dA: Pg_dtc732_20dA: Bth584_16dA = 1: 2.05: 0.39: 0.36: 0.19. CF were obtained using clones from bacterial strains that includes B. nordii (JCM12987), B. salyersiae (JCM12988), B. pyogenes (JCM6294) and B. thetaiotaomicron (BCRC10624).

TABLE 6
Comparison of the abundance of the Bacteroides at different hierarchical levels,
obtained from different published literature

| | | Sampling | | B. fragilis | B. distasonis | | B. | | |
|---|---|---|---|---|---|---|---|---|---|
| | Method | size | Bacteroides | subgroup | subgroup | B. vulgatus | thetaiotaomicron | B. uniformis | B. caccae |
| Franks et | FISH | Nine | N.A. | 21% w.r.t. Eub338- | | | Not tested | | |
| al. (12) | | volunteers, | | related domain | | | | | |
| | | eight fecal | | | | | | | |
| | | samples each | | | | | | | |
| Harmsen | FISH | 11 | 27.7% | Not tested | | | | | |
| et al. (14) | | volunteers, | (Bacteroidales | | | | | | |
| | | fecal | order) | | | | | | |
| | | samples | | | | | | | |
| Suau et | 16S rRNA | One | 31% | 13.3% of | 2.3% of | 2.1% of | 1.4% of | 4.9% of | 1.1% of |
| al. (31) | clone | volunteer, | (Bacteroides- | Bacteria | Bacteria | Bacteria | Bacteria | Bacteria | Bacteria |
| | libraries | fecal | Prevotella) | | | | | | |
| | | samples, | | | | | | | |
| | | 284 clones | | | | | | | |
| Eckburg et | 16S rRNA | Three | 48% | Not tested | | 15% of | 6.2% of | Not tested | |
| al. (7) | clone | volunteers, | (Bacteroidetes | | | | Bacteria | Bacteria | |
| | libraries | pool of 395 | phylum) | | | | | | |
| | | bacterial | | | | | | | |
| | | phylotypes | | | | | | | |
| | | from mucosa | | | | | | | |
| | | and feces | | | | | | | |
| Wang et | 16S rRNA | Two | 27.7% | Not tested | | | | | |
| al. (33) | clone | volunteers, | (Bacteroidetes | | | | | | |
| | libraries | colon | phylum) | | | | | | |
| | | samples | | | | | | | |
| This study | HOPE | Three | 14.3-21.8% | 3.0-6.6% | | 0.8-3.4% | 0.1-0.4% | 0.3-0.5% | 0.1-0.3% |
| | | volunteers, | (Bacteroidales | w.r.t. U1390- | | w.r.t. U1390- | w.r.t. U1390- | w.r.t. U1390- | w.r.t. |
| | | fecal | order) | related domain | | related domain | related domain | related domain | U1390- |
| | | samples | | | | | | | related |
| | | | | | | | | | domain |

(12) Franks, et al. 1998. Variations of bacterial populations in human feces measured by fluorescent in situ hybridization with group-specific 16S rRNA-targeted oligonucleotide probes. Appl Environ Microbiol 64: 3336-45.
(14) Harmsen, et al. 2002. Extensive set of 16S rRNA-based probes for detection of bacteria in human feces. Appl Environ Microbiol 68: 2982-90.
(31) Suau, et al. 1999. Direct analysis of genes encoding 16S rRNA from complex communities reveals many novel molecular species within the human gut. Appl Environ Microbiol 65: 4799-807.
(7) Eckburg, et al. 2005. Diversity of the human intestinal microbial flora. Science 308: 1635-8.
(33) Wang, et al. 2003. Molecular characterization of the microbial species that colonize human ileal and colonic mucosa by using 16S rDNA sequence analysis. J Appl Microbiol 95: 508-20.

1. A method for identifying target nucleic acids comprising the steps of:
(a) contacting a sample containing a plurality of target nucleic acids with at least one series of nucleotide primers under conditions that allow binding of said primers to at least one of said target nucleic acids and labeling of said bound primers with a detectable signal, wherein one member within each series has a lower level of specificity than other members of the series; and
(b) measuring said detectable signal of each labeled primer to determine the identity of said target nucleic acids.

2. The method of claim 1, wherein said sample comprises amplified said plurality of target nucleic acids.

3. The method of claim 1, wherein said plurality of target nucleic acids comprises 16S ribosomal RNA.

4. The method of claim 1, further comprising the step of purifying said labeled primers prior to step (b).

5. The method of claim 4, wherein said purification step is selected from the group consisting of a chromatographic separation, an enzyme-based digestion, spin column purification, chemical precipitation or an electrophoresis separation.

6. The method of claim 1, wherein each of said at least one series comprises between 3 and 20 nucleotide primers.

7. The method of claim 1, wherein at least one of said nucleotide primers within a series has a different length to other members of said series.

8. The method of claim 1, wherein each of said nucleotide primers within a series has a different length to other members of said series.

9. The method of claim 7, wherein said different lengths comprise differences of from 2 to 70 nucleotides.

10. The method of claim 7, wherein said different lengths are obtained using nucleic acid tails of different lengths.

11. The method of claim 10, wherein said nucleic acid tails are selected from the group consisting of poly dA tails and poly dT tails.

12. The method of claim 10, wherein said nucleic acid tails for individual members of a series range from 2 to 50 nucleotides.

13. The method of claim 1, wherein after said nucleotide primer is bound to said target nucleic acid, said method comprises the step of extending said primer with a single nucleotide labeled with a detectable signal.

14. The method of claim 13, wherein said single nucleotide is selected from the group consisting of the dideoxynucleoside triphosphates ddATP, ddTTP, ddCTP and ddGTP.

15. The method of claim 1, wherein said detectable signal is selected from the group consisting of a fluorescent signal, a chemiluminescent signal, a luminescent signal, a radioactive signal or a biotinylated signal.

16. The method of claim 15, wherein said fluorescent signal is selected from the group consisting of fluorescein, rhodamine, coumarin, cyanine, fluorescent nanoparticles and fluorescent guantum dyes.

17. The method of claim 1, wherein said detectable signal of a labeled primer is quantified by application of a calibration factor of one or more target nucleic acids.

18. The method of claim 17, wherein said calibration factor is obtained by determining the ratio of the intensity of a detectable signal of a bound primer having a higher level of specificity relative to the intensity of a detectable signal of a bound primer having a lower level of specificity.

19. The method of claim 1 comprising, prior to step (b), the step of separating said labeled primers to enable determination of the identity of said target nucleic acids.

20. The method of claim 19, wherein said separation step is selected from the group consisting of electrophoresis, chromatography and mass spectrometry.

21. The method of claim 1, wherein said conditions that allow binding of said primers to at least one of said target nucleic acids and labeling of said bound primers with a detectable signal comprise conditions for annealing of said primers to said target nucleic acids and extension of said primers with at least one single nucleotide labeled with a detectable signal, and multiple cycles thereof.

22. The method of claim 1, where step (a) comprises contacting a first aliquot of said sample with a first series of nucleotide primers and separately contacting a second aliquot of said sample with a second series of nucleotide primers, wherein said first and second series include at least one nucleotide primer in common.

23. The method of claim 1, wherein said method is a method of determining constituent organisms in a heterogenous sample.

24. The method of claim 23, wherein said constituent organisms are selected from the group consisting of prokaryotes and eukaryotes.

25. The method of claim 24, wherein said prokaryotes are selected from the group consisting of bacteria and archae.

26. The method of claim 24, wherein said eukaryotes are selected from the group consisting of fungi and yeast.

27. The method of claim 1, wherein said series of nucleotide primers comprises a plurality of nucleotide primers having a hierarchical level of specificity.

28. The method of claim 1, wherein said series comprises a primer at a higher hierarchical level and a plurality of primers at a lower hierarchical level.

29. The method of claim 1, wherein said series of nucleotide primers comprises a plurality of species-specific primers and a non-species-specific primer.

30. The method of claim 27, wherein said series comprises a domain-specific primer and a plurality of primers selected from the group consisting of phylum-specific primers, class-specific primers, order-specific primers, family-specific primers, genus-specific primers and species-specific primers.

31. The method of claim 27, wherein said series comprises a phylum-specific primer and a plurality of primers selected from the group consisting of class-specific primers, order-specific primers, family-specific primers, genus-specific primers and species-specific primers.

32. The method of claim 27, wherein said series comprises a class-specific primer and a plurality of primers selected from the group consisting of order-specific primers, family-specific primers, genus-specific primers and species-specific primers.

33. The method of claim 27, wherein said series comprises an order-specific primer and a plurality of primers selected from the group consisting of family-specific primers, genus-specific primers and species-specific primers.

34. The method of claim 27, wherein said series comprises a family-specific primer and a plurality of primers selected from the group consisting of genus-specific primers and species-specific primers.

35. The method of claim 27, wherein said series comprises a genus-specific primer and a plurality of species-specific primers.

36. The method of claim 1, wherein each member of a series, other than said one member with the lower level of specificity, is specific for one target nucleic acid.

37. The method of claim 1, wherein, at least one nucleotide primer within a series is capable of binding to multiple target nucleic acids.

38. A kit for use in a method for identifying target nucleic acids, the kit comprising:
(i) at least one series of nucleotide primers capable of binding to a plurality of target nucleic acids, wherein one member within each series has a lower level of specificity than other members of the series; and
(ii) instructions for contacting a sample containing a plurality of nucleic acids with said nucleotide primers under conditions that allow binding of said primers to at least one of said target nucleic acids and labeling of said bound primers with a detectable signal to allow measurement of said detectable signal of each labeled primer to determine the identity of said target nucleic acids.

39. The kit of claim 38, further comprising a set of labels having a detectable signal capable of binding to said nucleotide primers.

40. The kit of claim 38, wherein at least one of said nucleotide primers within a series has a different length to other members of said series.

41. The kit of claim 38, wherein each of said nucleotide primers within a series has a different length to other members of said series.

42. A method for diagnosis of a disorder of the gut in a subject, wherein said disorder is associated with an abnormal distribution of gut flora, said method comprising identifying target nucleic acids in a sample from said subject, said identifying comprising the steps of:
(a) contacting said sample containing target nucleic acids with at least one series of nucleotide primers under conditions that allow binding of said primers to at least one of said target nucleic acids and labeling of said bound primers with a detectable signal, wherein one member within each series has a lower level of specificity than other members of the series; and
(b) measuring said detectable signal of each labeled primer to determine the identity of said target nucleic acids, wherein said identification provides a determination of gut flora distribution.


SEQUENCE LISTING (65 sequences)

SEQ ID NO: 1 - DNA, 17 bp, Artificial Sequence
  Oligonucleotide Primer (11F)
gtttgatcctggctcag

SEQ ID NO: 2 - DNA, 19 bp, Artificial Sequence
  Oligonucleotide Primer (1492R)
ggntaccttgttacgactt

SEQ ID NO: 3 - DNA, 19 bp, Artificial Sequence
  Oligonucleotide Reverse Primer (1492R)
ggytaccttgttacgactt

SEQ ID NO: 4 - DNA, 18 bp, Artificial Sequence
  Oligonucletodie Primer (EUB338)
gctgcctcccgtaggagt

SEQ ID NO: 5 - DNA, 22 bp, Artificial Sequence
  Oligonucleotide Primer (EUB338-4a)
aaaagctgcctcccgtaggagt

SEQ ID NO: 6 - DNA, 26 bp, Artificial Sequence
  Oligonucleotide Primer (EUB338-8a)
aaaaaaaagctgcctcccgtaggagt

SEQ ID NO: 7 - DNA, 30 bp, Artificial Sequence
  Oligonucleotide Primer (EUB338-12a)
aaaaaaaaaaaagctgcctcccgtaggagt

SEQ ID NO: 8 - DNA, 36 bp, Artificial Sequence
  Oligonucleotide Primer (EUB338-18a)
aaaaaaaaaaaaaaaaaagctgcctcccgtaggagt

SEQ ID NO: 9 - DNA, 42 bp, Artificial Sequence
  Oligonucleotide Primer (EUB338-24a)
aaaaaaaaaaaaaaaaaaaaaaaagctgcctcccgtaggagt

SEQ ID NO: 10 - DNA, 48 bp, Artificial Sequence
  Oligonucleotide Primer (EUB338-30a)
aaaaaaaaaaaaaaaaaaaaaaaaaaaaaagctgcctcccgtaggagt

SEQ ID NO: 11 - DNA, 17 bp, Artificial Sequence
  Oligonucleotide Primer (EUB338Ia)
gctgcctcccgtaggag

SEQ ID NO: 12 - DNA, 40 bp, Artificial Sequence
  Oligonucleotide Primer (EUB338Ia-23a)
aaaaaaaaaaaaaaaaaaaaaaagctgcctcccgtaggag

SEQ ID NO: 13 - DNA, 22 bp, Artificial Sequence
  Oligonucleotide Primer (BAC303-5a)
aaaaaccaatgtgggggacctt

SEQ ID NO: 14 - DNA, 32 bp, Artificial Sequence
  Oligonucleotide Primer (BTH274-15a)
aaaaaaaaaaaaaaacccctatccatcgaagg

SEQ ID NO: 15 - DNA, 36 bp, Artificial Sequence
  Oligonucleotide Primer (BTH584-16a)
aaaaaaaaaaaaaaaacaactgacttaactgtccac

SEQ ID NO: 16 - DNA, 38 bp, Artificial Sequence
  Oligonucleotide Primer (BFRG602-19a)
aaaaaaaaaaaaaaaaaaagagccgcaaactttcacaa

SEQ ID NO: 17 - DNA, 18 bp, Artificial Sequence
  Oligonucleotide Primer (BTT1250)
cgccgtgtagcaacctgc

SEQ ID NO: 18 - DNA, 19 bp, Artificial Sequence
  Oligonucleotide Primer (BFRG1024)
tcacagcggtgattgctca

SEQ ID NO: 19 - DNA, 18 bp, Artificial Sequence
  Oligonucleotide Primer (BITT141)
cgaaaggctatcccggaa

SEQ ID NO: 20 - DNA, 27 bp, Artificial Sequence
  Oligonucleotide Primer (BADF1037-9a)
aaaaaaaaatgcagcaccttcacacct

SEQ ID NO: 21 - DNA, 37 bp, Artificial Sequence
  Oligonucleotide Primer (BUFM1018-18a)
aaaaaaaaaaaaaaaaaaaactgccttgcggctgaca

SEQ ID NO: 22 - DNA, 16 bp, Artificial Sequence
  Fluorophore-labeled Oligonucleotide Standard (GT16-Cy5)
gtgtgtgtgtgtgtgt

SEQ ID NO: 23 - DNA, 26 bp, Artificial Sequence
  Fluorophore-labeled Oligonucleotide Standard (GT26-Cy5)
gtgtgtgtgtgtgtgtgtgtgtgtgt

SEQ ID NO: 24 - DNA, 36 bp, Artificial Sequence
  Fluorophore-labeled Oligonucleotide Standard (GT36-Cy5)
gtgtgtgtgtgtgtgtgtgtgtgtgtgtgtgtgtgt

SEQ ID NO: 25 - DNA, 46 bp, Artificial Sequence
  Fluorophore-labeled Oligonucleotide Standard (GT46-Cy5)
gtgtgtgtgtgtgtgtgtgtgtgtgtgtgtgtgtgtgtgtgtgtgt

SEQ ID NO: 26 - DNA, 46 bp, Artificial Sequence; identical to SEQ ID NO: 25
  Fluorophore-labeled Oligonucleotide Standard (GT46-D2)

SEQ ID NO: 27 - DNA, 40 bp, Artificial Sequence
  Fluorophore-labeled Oligonucleotide Standard (GT40-D4)
gtgtgtgtgtgtgtgtgtgtgtgtgtgtgtgtgtgtgtgt

SEQ ID NO: 28 - DNA, 38 bp, Artificial Sequence
  Oligonucleotide Primer (Pg_dtc732)
aaaaaaaaaaaaaaaaaaaagttccggcccggtgagct

SEQ ID NO: 29 - DNA, 27 bp, Artificial Sequence
  Oligonucleotide Primer (Bhcg 171)
aaaaaaaatttcagtgccatcgggcat

SEQ ID NO: 30 - DNA, 34 bp, Artificial Sequence
  Oligonucleotide Primer (Bcc 1066)
aaaaaaaaaaaaaaacgtatgggtttccccataa

SEQ ID NO: 31 - DNA, 18 bp, Artificial Sequence
  Oligonucleotide Primer (Badf 1009)
cggctaacatgtttccac

SEQ ID NO: 32 - DNA, 19 bp, Artificial Sequence
  Oligonucleotide Primer (Bvg 1016)
atgccttgcggcttacggc

SEQ ID NO: 33 - DNA, 43 bp, Artificial Sequence
  Oligonucleotide Primer (Bcpc 1015)
aaaaaaaaaaaaaaaaaaaaaaaacgccttgcggcttacaagt

SEQ ID NO: 34 - DNA, 38 bp, Artificial Sequence
  Oligonucleotide Primer (Bmsl 1000)
aaaaaaaaaaaaaaaaaaagcgtttccgccatattcgg

SEQ ID NO: 35 - DNA, 18 bp, Artificial Sequence
  Oligonucleotide Primer (Begt 999)
gtttccactacattccgc

SEQ ID NO: 36 - DNA, 19 bp, Artificial Sequence
  Oligonucleotide Primer (Bnd 136)
agcctatccccgagtaaaa

SEQ ID NO: 37 - DNA, 27 bp, Artificial Sequence
  Oligonucleotide Primer (Bsls 1016)
aaaaaaaaaagccttgcggctatgccg

SEQ ID NO: 38 - DNA, 18 bp, Artificial Sequence
  Oligonucleotide Primer (Bmde657)
tccgcctacctcaaacac

SEQ ID NO: 39 - DNA, 26 bp, Artificial Sequence
  Oligonucleotide Primer (Bgld277)
aaaaaaaagaacccctatccatcgtg

SEQ ID NO: 40 - DNA, 18 bp, Artificial Sequence
  Oligonucleotide Primer (Bdts1278)
agacgtggtttggggatt

SEQ ID NO: 41 - DNA, 37 bp, Artificial Sequence; identical to SEQ ID NO: 21
  Oligonucleotide Primer (Bufm 1018)

SEQ ID NO: 42 - DNA, 38 bp, Artificial Sequence
  Oligonucleotide Primer (Bfrg 1026)
aaaaaaaaaaaaaaaaaaaatcacagcggtgattgctc

SEQ ID NO: 43 - DNA, 29 bp, Artificial Sequence
  Oligonucleotide Primer (Bdts_gp980)
aaaaaaaaaaaacgttcaaacccgggtaa

SEQ ID NO: 44 - DNA, 32 bp, Artificial Sequence
  Oligonucleotide Primer (UI390)
aaaaaaaaaaaaaaaaaygacgggcggtgtgt

SEQ ID NO: 45 - DNA, 49 bp, Escherichia coli
gactcctacgggaggcagcagtggggaatattgcacaatgggcgcaagc

SEQ ID NO: 46 - DNA, 19 bp, B. thetaiotaomicron
aactcctacgggaggcagc

SEQ ID NO: 47 - DNA, 19 bp, B. fragilis
aactcctacgggaggcagc

SEQ ID NO: 48 - DNA, 19 bp, B. vulgatus
aactcctacgggaggcagc

SEQ ID NO: 49 - DNA, 19 bp, B. distasonis
aactcctacgggaggcagc

SEQ ID NO: 50 - DNA, 18 bp, B. thetaiotaomicron
gaaggtcccccacattgg

SEQ ID NO: 51 - DNA, 18 bp, B. fragilis
gaaggtcccccacattgg

SEQ ID NO: 52 - DNA, 18 bp, B. vulgatus
gaaggtcccccacattgg

SEQ ID NO: 53 - DNA, 18 bp, B. distasonis
gaaggtcccccacattgg

SEQ ID NO: 54 - DNA, 18 bp, B. thetaiotaomicron
accttcgatggatagggg

SEQ ID NO: 55 - DNA, 18 bp, B. fragilis
gccttcgatggatagggg

SEQ ID NO: 56 - DNA, 18 bp, B. vulgatus
gtcttcgatggatagggg

SEQ ID NO: 57 - DNA, 18 bp, B. distasonis
accgacgatggatagggg

SEQ ID NO: 58 - DNA, 21 bp, B. thetaiotaomicron
ggtggacagttaagtcagttg

SEQ ID NO: 59 - DNA, 21 bp, B. fragilis
ggtggactggtaagtcagttg

SEQ ID NO: 60 - DNA, 21 bp, B. vulgatus
gatggatgtttaagtcagttg

SEQ ID NO: 61 - DNA, 21 bp, B. distasonis
ggcggccttttaagtcagcgg

SEQ ID NO: 62 - DNA, 19 bp, Artificial Sequence
  Probe EUB338
gctgcctcccgtaggagtt

SEQ ID NO: 63 - DNA, 18 bp, Artificial Sequence
  Probe BAC303-5a
ccaatgtgggggaccttc

SEQ ID NO: 64 - DNA, 18 bp, Artificial Sequence
  Probe BTH274-15a
cccctatccatcgaaggt

SEQ ID NO: 65 - DNA, 21 bp, Artificial Sequence
  Probe BTH584-16a
caactgacttaactgtccacc